SEQUENCE LISTING (13 sequences)

SEQ ID NO: 1 - protein, 92 aa, Artificial sequence
  Synthetic peptide
AVRQKRRIYDITNVLEGIDLIEKKSKNSIQWKGVGAGCNTKEVIDRLRYLKAEIEDLELKERELDQQKLLLQQSIKNVMDDSINNRFSYVTH

SEQ ID NO: 2 - protein, 32 aa, Artificial sequence
  Synthetic peptide
ELDQQKLWLQQSIKNVMDDSINNRFSYVTHED

SEQ ID NO: 3 - protein, 33 aa, Artificial sequence
  Synthetic peptide
LILFVIKLHQDVNDMRNSNQDQTQSEDRESKWY

SEQ ID NO: 4 - RNA, 21 nt, Artificial sequence
  Synthetic oligonucleotide
aggaggaugaaauagaugguu

SEQ ID NO: 5 - DNA, 35 bp, Artificial sequence
  Synthetic oligonucleotide
gaggtggaggtctagatcaccaatgtcttagaggg

SEQ ID NO: 6 - DNA, 35 bp, Artificial sequence
  Synthetic oligonucleotide
cagtgtggtggaattctatgtatcaccatgaaagc

SEQ ID NO: 7 - protein, 8 aa, Artificial sequence
  Synthetic peptide
RRRRRRRR

SEQ ID NO: 8 - protein, 13 aa, Artificial sequence
  Synthetic peptide
GRKKRRQRRRPPQ

SEQ ID NO: 9 - protein, 9 aa, Artificial sequence
  Synthetic peptide
RKKRRQRRR

SEQ ID NO: 10 - protein, 16 aa, Artificial sequence
  Synthetic peptide
RQPKIWFPNRRKPWKK

SEQ ID NO: 11 - protein, 16 aa, Artificial sequence
  Synthetic peptide
RQIKIWFQNRRMKWKK

SEQ ID NO: 12 - protein, 26 aa, Artificial sequence
  Synthetic peptide
GIGAVLKVLTTGLPALISWIKRKRQQ

SEQ ID NO: 13 - protein, 346 aa, Homo sapiens
MAAAEPASSGQQAPAGQGQGQRPPPQPPQAQAPQPPPPPQLGGAGGGSSRHEKSLGLLTTKFVSLLQEAKDGVLDLKAAADTLAVRQKRRIYDITNVLEGIDLIEKKSKNSIQWKGVGAGCNTKEVIDRLRYLKAEIEDLELKERELDQQKLLLQQSIKNVMDDSINNRFSYVTHEDICNCFNGDTLLAIQAPSGTQLEVPIPEMGQNGQKKYQINLKSHSGPIHVLLINKESSSSKPVVFPVPPPDDLTQPSSQSLTPVTPQKSSMATQNLPEQHVSERSQALQQTSATDISSAGSISGDIIDELMSSDVFPLLRLSPTPADDYNFNLDDNEGVCDLFDVQILNY

(19) United States
(12) Patent Application Publication
(10) Pub. No.: US 2019/0255146 A1
(43) Pub. Date: Aug. 22, 2019

(54) POLYPEPTIDES FOR IMPROVED RESPONSE TO ANTI-CANCER THERAPY

(71) Applicant: MUSC FOUNDATION FOR RESEARCH DEVELOPMENT, Charleston, SC (US)

(72) Inventors: Besim OGRETMEN, Mount Pleasant, SC (US); Raquela J. THOMAS, Charleston, SC (US); Natalia OLEINIK, Charleston, SC (US)

(73) Assignee: MUSC FOUNDATION FOR RESEARCH DEVELOPMENT, Charleston, SC (US)

(21) Appl. No.: 16/344,049

(22) PCT Filed: Oct. 30, 2017
(86) PCT No.: PCT/US17/58974

§ 371 Date: Apr. 23, 2019

Related U.S. Application Data
(60) Provisional application No. 62/415,122, filed on Oct. 31, 2016.

(51) Int. Cl. A61K 38/17; A61P 35/00; A61K 31/164; A61K 9/00; A61K 31/282

(52) U.S. Cl. CPC A61K 38/1709; A61P 35/00; A61K 31/164; A61K 45/06; A61K 9/0073; A61K 31/282; A61K 9/0019

(57) ABSTRACT

The present disclosure provides E2F5 mimetic polypeptides. Further provided are methods for the treatment of cancer, such as head and neck cancer, comprising administering the E2F5 mimetic polypeptides alone or in combination with an additional anti-cancer therapy, such as a chemotherapeutic.

This application claims the benefit of U.S. Provisional Patent Application No. 62/415,122, filed Oct. 31, 2016, the entirety of which is incorporated herein by reference.

The invention was made with government support under Grant Nos. DE016572-0751 and 5T32-DE017551 awarded by the National Institutes of Health. The government has certain rights in the invention.

### INCORPORATION OF SEQUENCE LISTING

The sequence listing that is contained in the file named “MESCP0101USP1_ST25.txt”, which is 7 KB (as measured in Microsoft Windows®) and was created on Oct. 30, 2017, is filed herewith by electronic submission and is incorporated by reference herein.

### BACKGROUND OF THE INVENTION

### 1. Field of the Invention

The present invention relates generally to the fields of molecular biology and medicine. More particularly, it concerns an E2F5 polypeptide and its use thereof for the treatment of cancer.

### 2. Description of Related Art

Human papillomavirus (HPV) infection is linked with several cancers such as cervix and head and neck carcinomas (Killock et al., 2015; Lehtinen et al., 2013). The potential impact of prophylactic HPV vaccines on the prevention of these cancers is of interest. However, there are differences in the epidemiology of oral and genital HPV infection, such as differences in age and sex distributions, which suggest that the vaccine efficacy observed in genital cancers may not be directly translatable to HPV-positive head and neck cancers, which are mainly located in the oropharynx (Leemans et al., 2011; D'Souza et al., 2007). Paradoxically, HPV infection is associated with improved survival outcome in response to chemo-radiotherapy for patients with head and neck squamous cell carcinoma (HNSCC), and not in patients with HPV-positive cervical cancers (Ang et al., 2010; Fakhry et al., 2008). However, molecular mechanisms involved in increased HNSCC cell death by HPV signaling in response to therapy are largely unknown.

A novel form of cell death, lethal mitophagy, was recently identified in HPV-negative HNSCC, induced by pro-cell death signaling sphingolipid molecule ceramide (Sentelle et al., 2012). Dynamin-related protein 1 (Drp1) activation is an upstream inducer of ceramide-dependent lethal mitophagy. Upon cellular stress, Drp1 translocates to the outer mitochondrial membrane, where it associates with the mitochondrial fission factor receptor (MFF) and forms oligomers, resulting in mitochondrial fission, leading to targeting of damaged mitochondria by autophagosomes (Strack and Cribbs, 2012; Smirnova et al., 2001). Drp1-mediated mitochondrial fission mediates direct lipid-protein interaction between ceramide accumulated in the outer mitochondrial membrane and microtubule associated protein 1 light chain 3 beta (LC3B), a component of autophagosomes, for selectively targeting/degradation of mitochondria by autophagy (mitophagy) (Dany and Ogretmen, 2015). In some cancer cells, such as HNSCC, targeting mitochondria by mitophagy leads to cell death due to decreased cellular energy and/or reduced mitochondrial metabolism/signaling, altering the production of molecules that are essential for cellular growth including nucleotides, amino acids and metabolic intermediates (Weinberg and Chandel, 2015).

However, whether induction of ceramide-mediated lethal mitophagy is involved in increased cell death in HPV-positive HNSCC in response to therapeutic treatment and cellular stress has not been described previously. Therefore, there is an unmet to investigate the role and mechanisms by which HPV signaling enhances the response of HNSCC versus cervical cancer cells to chemotherapy-mediated cellular stress by ceramide-dependent lethal mitophagy for the development of improved cancer therapeutics.

### SUMMARY OF THE INVENTION

Embodiments of the present disclosure provide E2F5 mimetic peptides, particularly a fragment of an E2F5 polypeptide comprising a biologically active dimerization domain of E2F5. Thus, in a first embodiment, there is provided an isolated polypeptide comprising an amino acid sequence having at least 85% sequence identity to SEQ ID NO: 1 or SEQ ID NO: 2, wherein the polypeptide comprises less than 100 contiguous amino acids of a E2F5 polypeptide (provided as SEQ ID NO: 13). In some aspects, a polypeptide of the embodiments selectively binds dynamin related protein 1 (Drp1).

Thus in a further aspect, an E2F5 polypeptide of the embodiments comprises a sequence at least 90%, 91%, 92%, 93% 94%, 95%, 96%, 97%, 98%, 99%, or 100% identical to SEQ ID NO: 1, wherein the sequence comprises less than less than 100 or less than 95 contiguous amino acids of an E2F5 polypeptide (provided as SEQ ID NO: 13). In certain aspects, the polypeptide is no more than 100 or 95 amino acids in length.

In still a further aspect, an E2F5 polypeptide of the embodiments comprises a sequence at least 90%, 91%, 92%, 93% 94%, 95%, 96%, 97%, 98%, 99%, or 100% identical to SEQ ID NO: 2, wherein the sequence comprises less than less than 100, 95, 85, 80, 75, 70, 65, 60, 55, 50, 45, 49, 48, 47, 46, 40, 39, 38, 37, 36, 35, 34, 33, 32, 31, or 30 contiguous amino acids of SEQ ID NO: 13. In certain aspects, the polypeptide is no more than 100, 95, 85, 80, 75, 70, 65, 60, 55, 50, 45, 49, 48, 47, 46, 40, 39, 38, 37, 36, 35, 34, 33, 32, 31, or 30 amino acids in length.

In certain aspects, the polypeptide further comprises a cell penetration sequence, a cell targeting sequence or a stabilization sequence. For example, the cell targeting sequence can be a cell binding peptide or an antibody domain. In some aspects, the cell penetration sequence is a polyarginine sequence, such as RRRRRRRR.

A further embodiment provides a pharmaceutical composition comprising the isolated polypeptide of the embodiments (e.g., comprising an amino acid sequence having at least 90% sequence identity to SEQ ID NO:2) and a pharmaceutical carrier. In some aspects, the pharmaceutical composition is formulated for parenteral administration, intravenous injection, intramuscular injection, inhalation, or subcutaneous injection. Also provided herein is an isolated nucleic acid encoding the polypeptide of the embodiments. In some aspects, the nucleic acid is comprised in a vector, such as a mammalian expression vector viral vector. Another embodiment provides a host cell comprising the nucleic acid encoding the polypeptide of the embodiments.

In another embodiment, there is provided a method for treating cancer in a subject comprising administering an effective amount of the polypeptide of the embodiments (e.g., comprising an amino acid sequence having at least 90% sequence identity to SEQ ID NO:2) to the subject. In some aspects, the cancer is head and neck cancer. In certain aspects, the subject is diagnosed as Human papillomavirus (HPV) negative.

In additional aspects, the method further comprises administering at least a second anti-cancer therapy. In some aspects, the second anti-cancer therapy is selected from the group consisting of a chemotherapy, a radiotherapy, an immunotherapy, or a surgery. In particular aspects, the chemotherapy is cisplatin. In one specific aspect, the at least a second anti-cancer therapy is a ceramide analogue drug, such as C₁₈-pyridinium-ceramide (C₁₈-pyr-cer). In some aspects, the polypeptide enhances ceramide-induced Drp1 recruitment to mitochondria. In certain aspects, the polypeptide enhances chemotherapy-induced cell death and/or mitophagy.

Other objects, features and advantages of the present invention will become apparent from the following detailed description. It should be understood, however, that the detailed description and the specific examples, while indicating certain embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-D. HPV-mediated HNSCC cell death is CerS1/ceramide dependent. (A) Involvement of CerS1/ceramide pathway in the induction of mitophagy was assessed using live cell imaging in MEFs isolated from WT or CerS1^{top/top} mice in the absence/presence of known mitophagy inducer sodium selenite. (B) Effects of reconstitution of CerS1^{WT} versus catalytically inactive mutant CerS1^{H183A} in CerS1^{top/top} MEFs on mitophagy in the absence/presence of sodium selenite were measured by live cell imaging. In A-B, images were quantified by ImageJ, and data are means±SD from three independent experiments. *p<0.05 as determined by Student's t test. (C-D) Effects of shRNA-mediated knockdown of CerS1, confirmed by Western blotting (C), on cisplatin-mediated cell death were measured by MTT assay (D). Data are means±SD from three independent experiments. *p<0.05 as determined by Student's t test.

FIGS. 2A-G. HPV-E6 enhances chemotherapy-mediated cellular stress and CerS1/ceramide-mediated lethal mitophagy. (A-B) Effects of siRNA-mediated knockdown of HPV-E6/E7 on HPV(+) UM-SCC-47 cell death was assessed by MTT compared to Scr-siRNA-transfected and/or vehicle-treated controls. Successful knockdown of HPV-E6/E7 proteins were confirmed by Western blot analysis (B). Data are means±SD from three independent experiments. *p<0.05 as determined by Student's t test. (C) Effects of ectopic expression of HPV-E6 versus E7 on HPV(−) UM-SCC-22A cell death were measured by MTT. HPV-E6/E7 protein abundance was measured by Western blotting. Data are means±SD from three independent experiments. *p<0.05 as determined by Student's t test. (D) Effects of shRNA-mediated knockdown of CerS1 on mitophagy in response to cisplatin were measured by live cell imaging confocal micrographs of UM-SCC-47 cells stained with LTG and MTR. Scr-shRNA-transfected and/or vehicle-treated cells were used as controls. Data are means±SD from three independent experiments. *p<0.05 as determined by Student's t test. Images were quantified using ImageJ. (E) Cytoplasmic versus mitochondrial localization of CerS1 in the presence/absence of cisplatin at 0, 1, and 4 h was measured by Western blotting using anti-CerS1 antibody in cytoplasm (cyto)—versus mitochondria (mito)-enriched cellular fractions of UM-SCC-47 cells. Actin and Tom 20 were used as controls for cytoplasm- and mitochondria-enriched fractions, respectively. In Western blot panels, images are representative of three independent experiments. Western blot images were quantified using ImageJ and *p<0.05 as determined by Student's t test (lower panel). (F-G) Effects of shRNA-mediated knockdown of CerS6, confirmed by Western blotting (F), on UM-SCC-47 cell death in response to cisplatin were measured by trypan blue exclusion assay (G). Data are means±SD from three independent experiments. *p<0.05 as determined by Student's t test. In Western blots (F), actin was used as a loading control.

FIGS. 3A-E. Mitochondrial targeting of ceramide induces lethal mitophagy in HPV(+) cells. (A) Mitophagy was measured in response to C₁₈-pyr-cer in UM-SCC-47 cells using Sea Horse for the detection of mitochondrial respiration (oxygen consumption rate) compared to vehicle-treated controls. Data are means±SD from three independent experiments. *p<0.05 as determined by Student's t test. (B) Effects of C₁₈-pyr-cer in the induction of mitophagy in HPV(−) UM-SCC-22A versus HPV(+) UM-SCC-47 cells were measured by TEM. Number of mitochondria and autophagosomes were counted in TEM micrographs (right panel). Data are means±SD from three independent experiments. *p<0.05 as determined by Student's t test. (C-D) Mitophagy was detected in response to C₁₈-pyr-cer at 0-120 min exposure using live cell imaging confocal micrographs of cells stained with LTG and MTR in the presence of shRNA-mediated knockdown of ATG5 or LC3B compared to Scr-shRNA-transfected controls (C). Images were quantified using ImageJ (D). Data are means±SD from three independent experiments. *p<0.05 as determined by Student's t test. (E) UM-SCC-47 cell death was measured by trypan blue exclusion assay in the presence of Scr-siRNA or ATGS-siRNA in response to C₁₈-pyr-cer exposure. Data are means±SD from three independent experiments. *p<0.05 as determined by Student's t test.

FIGS. 4A-E. Mitochondrial ceramide is involved in HPV-E7-mediated lethal mitophagy. (A-B) Effects of siRNA-mediated knockdown of HPV-E6/E7 on mitophagy in response to C₁₈-pyr-cer, measured by live cell imaging confocal micrographs of UM-SCC-47 cells stained with LTG and MTR (A) or by mitochondrial respiration using the Sea Horse for measurement of oxygen consumption rate (B). Scr-siRNA-transfected and/or vehicle-treated cells were used as controls. Data are means±SD from three independent experiments. *p<0.05 as determined by Student's t test. (C-E) Roles of ectopic expression of HPV-E6 or E7 in UM-SCC-22A cells for enhancing mitochondrial C₁₈-pyr-cer-mediated cell death and mitophagy were measured by trypan blue exclusion assay (C) and Sea Horse for measuring mitochondrial respiration (oxygen consumption rate) (D) or using live cell imaging confocal micrographs of UM-SCC-22A cells stained with LTG and MTR (E). Vector-transfected and/or vehicle-treated cells were used as controls. Data are means±SD from three independent experiments. *p<0.05 as determined by Student's t test. Images were quantified using ImageJ.

FIGS. 5A-D. HPV-E7 targets RB for induction of ceramide-dependent mitophagy. (A) Roles of shRNA-mediated knockdown of p53, a target of HPV-E6, versus RB, a target of HPV-E7, on ceramide-mediated UM-SCC-22A cell death were measured by trypan blue exclusion assay. Scr-shRNA-transfected and/or vehicle-treated cells were used as controls. Data are means±SD from three independent experiments. *p<0.05 as determined by Student's t test. Protein abundance of p53 and RB were measured by Western blotting in cells transfected with shRNAs. Actin was used as a loading control (inset). Samples shown in RB Western blot (right panel, inset) are from the same representative blot but not in contiguous lanes. (B-D) Effects of ectopic expression of RB10 (active mutant of RB which is not recognized by HPV-E7) on UM-SCC-47 cell death and mitophagy were measured by trypan blue exclusion assay (B) and Sea Horse for detecting mitochondrial respiration (oxygen consumption rate) (C) or using live cell imaging confocal micrographs of UM-SCC-47 cells stained with LTG and MTR (D) in the absence or presence of C₁₈-pyr-cer. Vector-transected cells were used as controls Data are means±SD from three independent experiments. *p<0.05 as determined by Student's t test. Images were quantified using ImageJ.

FIGS. 6A-E. HPV-E7/RB axis regulates ceramide-dependent mitophagy via E2F5 activation. (A) UM-SCC-47 (HPV+) cell death was measured using trypan blue exclusion assay in response to shRNA-mediated knockdown of E2F1, E2F4 or E2F5 in response to C₁₈-pyr-cer compared to Scr-shRNA-transfected and/or vehicle-treated controls. Efficiency of shRNA-mediated knockdown of E2F1, E2F4 or E2F5 mRNAs was measured by RT-PCR (normalized to actin mRNA) compared to Scr-shRNA-transfected controls (right panel). Data are means±SD from three independent experiments. *p<0.05 as determined by Student's t test. (B) Effects of siRNA-mediated knockdown of HPVE6/E7 on endogenous RB-E2F5 association in UM-SCC-47 cells were measured by PLA using fluorescently labeled anti-RB and anti-E2F5 antibodies. Quantification of PLA signals were performed as described by the manufacturer using the PLA software. Data are means±SD from three independent experiments. *p<0.05 as determined by Student's t test. (C) Effects of shRNA-mediated E2F5 knockdown on mitophagy in response to vehicle versus C₁₈-pyr-cer were measured by live cell imaging confocal micrographs of UM-SCC-47 cells stained with LTG and MTR. Data are means±SD from three independent experiments. *p<0.05 as determined by Student's t test. Images were quantified using ImageJ. (D-E) Cell death or mitophagy were measured in response to ectopic expression of E2F5 in the presence/absence of C₁₈-pyr-ceramide by trypan blue exclusion assay (D) or by live cell imaging confocal micrographs of UM-SCC-22A cells stained with LTG and MTR (E). Vector-transfected cells were used as controls. Data are means±SD from three independent experiments. *p<0.05 as determined by Student's t test. Images were quantified using ImageJ. Protein abundance of E2F5 after transient transfections was measured by Western blotting. Actin was used as a loading control (D, lower panel).

FIGS. 7A-F. E2F5/Drp1 complex enhances HPV-E7/ceramide-dependent lethal mitophagy. (A) UM-SCC47 cells were treated with cisplatin or C₁₈-pyr-cer and Western blotting was performed with cell lysates in the absence of reducing agents in the lysing buffer to detect monomeric and dimeric Drp1 protein abundance compared to vehicle-treated controls. Actin was used as a loading control. (B-C) Cell death was assessed by trypan blue exclusion assay in UM-SCC47 cells expressing dominant-negative Drp1 mutant (K38A-DRP1) and treated with C₁₈-pyr-cer (B) or cisplatin (C) compared to vector-transfected controls. Data are means±SD from three independent experiments. *p<0.05 as determined by Student's t test. (D-E) Association between E2F5 and Drp1 in response to ceramide stress (C₁₈-pyr-cer) was measured in UM-SCC-47 cells using PLA (D) or immunoprecipitation followed by Western blotting (E) with anti-Drp1 or anti-E2F5 antibodies. IgG was used as a control. Data are means±SD from three independent experiments. *p<0.05 as determined by Student's t test. (F) Effect of siRNA-mediated knockdown of HPVE6/E7 on Drp1-E2F5 association in the absence/presence of ceramide stress (C₁₈-pyr-cer) was measured by PLA using fluorescently labeled anti-Drp1 and anti-E2F5 antibodies in HPV(+) UPI-SCC-90 cells. PLA signals were quantified as described by the manufacturer using the PLA quantification software. Data are means±SD from three independent experiments. *p<0.05 as determined by Student's t test. Effects of siRNA-mediated HPV-E6/E7 knockdown on pRB, HPV-E7 and p53 protein abundance were also confirmed by Western blotting compared to Scr-siRNA-transfected controls in the absence/presence of C₁₈-pyr-cer (right panel). Total RB was used a loading control (Rb band, upper panel).

FIGS. 8A-E. Activation of Drp1 by E2F5-Drp1 complex enhances HPV-E7/ceramide-mediated lethal mitophagy. (A) Top computer-generated model of E2F5 (orange)-Drp1 (grey) docking is shown, with the first and last residues of the known dimerization domain of E2F5 indicated, including residues 84 and 177 (left panel). Models of GFP-tagged wild type E2F5 (E2F5^{WT}) and dimerization domain deleted mutant of E2F5 (E2F5^{Δ84-177}) are shown (right panel). (B) UM-SCC22A cells transfected E2F5^{WT}-GFP or E2F5^{Δ84-177}-GFP were used for PLA to measure their association of RB using fluorescently labeled anti-RB and anti-GFP antibodies. PLA images were quantified using the PLA software as described by the manufacturer. Data are means±SD from three independent experiments. *p<0.05 as determined by Student's t test. (C-E) UM-SCC22A cells expressing E2F5^{WT} or mutant-E2F5^{Δ84-177} were used for the measurement of Drp1-E2F5 association in the absence/presence of ceramide stress using PLA (C) compared to vector-transfected controls. Transfection efficiency and abundance of ectopically expressed E2F5^{WT}-GFP and mutant-E2F5^{Δ84-177}-GFP was detected by immunofluorescence. Vector-GFP was used as a control (right panel, C). Moreover, effects of E2F5^{WT} versus mutant-E2F5^{Δ84-177} on cell death or mitophagy were measured by trypan blue exclusion assay (D) or by live cell imaging confocal micrographs (E) of UM-SCC-22A cells stained with LTR and MTB (mitotracker blue). Data are means±SD from three independent experiments. *p<0.05 as determined by Student's t test.

FIGS. 9A-D. E2F5 enhances Drp1 translocation to mitochondria and association with MFF to induce HPV-E7/ceramide-dependent mitophagy. (A) Effects of stable knockdown of E2F5 using shRNA on DRP1-MFF association with/without C₁₈-pyr-cer were measured by PLA using anti-DRP1 and anti-MFF antibodies compared to Scr-shRNA-transfected UM-SCC-47 controls. Data are means±SD from three independent experiments. *p<0.05 as determined by Student's t test. (B) HPV(−) UM-SCC1A cells transiently transfected with exogenous E2F5 or empty vector (vec) were used to measure the association between Drp1 and MFF in the absence/presence of ceramide stress by PLA using anti-DRP1 and anti-MFF antibodies. Data are means±SD from three independent experiments. *p<0.05 as determined by Student's t test. (C) Effects of shRNA-mediated E2F5 knockdown on Drp1 localization to mitochondria in the absence/presence of C₁₈-pyr-cer were assessed in whole cell lysates (UM-SCC-47) versus mitochondria-enriched fractions using Western blotting. (D) Effects of transient reconstitution of E2F5^{WT} or E2F5^{Δ84-177} proteins in UM-SCC47 cells, which were stably transfected with E2F5-shRNA, on Drp1 abundance, were measured by Western blotting using anti-Drp1 antibody, in whole cell lysates versus mitochondria-enriched fractions. Actin and Tom 20 were used as controls for whole cell and mitochondria-enriched fractions, respectively. In all Western blot panels, images are representative of three independent experiments.

FIGS. 10A-H. Reconstitution of Drp1-E2F5 binding using an E2F5 peptide mimetic enhances ceramide-mediated lethal mitophagy in HPV(−) HNSCC cells and tumor xenografts. (A-B) UM-SCC2A cells were treated with a peptide designed to mimic the putative Drp1-binding region of E2F5 (E2F5-pept) or scrambled control peptide (Scr-pept), and their effects on cell death in response to cisplatin (A) or C₁₈-pyr-cer (B) were measured by trypan blue exclusion assay. Data are means±SD from three independent experiments. *p<0.05 as determined by Student's t test. (C) Effects of shRNA-mediated knockdown of Drp1 on UM-SCC-22A cell death in the presence of E2F5-pept with/without C₁₈-pyr-cer were measured by trypan blue exclusion assay. Data are means±SD from three independent experiments. *p<0.05 as determined by Student's t test. (D) Live cell imaging in UM-SCC-22A cells treated with/without C₁₈-pyr-cer was performed to measure the effects of Scr-pept (control) versus E2F5-pept using live cell imaging confocal micrographs of UM-SCC-22A cells stained with LTR and MTG. Images were quantified using FIJI. Data are means±SD from three independent experiments. *p<0.05 as determined by Student's t test. (E) Drp1 localization to mitochondria in response to C₁₈-pyr-cer in the presence of Scr-pept versus E2F5-pept was measured by Western blotting using whole cell (UM-SCC-22A) lysates versus mitochondria-enriched fractions. Actin and Tom 20 were used as controls for whole cell and mitochondria-enriched fractions, respectively. In all Western blot panels, images are representative of three independent experiments. (F) Xenograft tumors generated from UM-SCC22A cells in the flanks of SCID mice were treated with cisplatin or DMSO (veh) in the presence of E2F5-pept or Scr-pept for 14 days, and tumor volumes were measured using calipers (n=5-8 mice/group, and *p<0.05). (G) Effects of E2F5-pept versus Scr-pept on mitophagy induction (by direct counting of mitophagosomes in TEM micrographs) in UM-SCC-22A xenograft-derived tumors isolated from SCID mice treated with Scr-peptide/vehicle, Scr-pept/cisplatin, E2F5-pept/vehicle and E2F5-pept/cisplatin were measured by TEM. Data are means±SD from three independent experiments. *p<0.05 as determined by Student's t test (n=8 images/group). (H) Association between Drp1 and E2F5 was determined by PLA in tumor tissue sections isolated from SCID mice treated with Scr-pept/vehicle or Scr-cisplatin versus E2F5-pept/vehicle or E2F5-pept-cisplatin by PLA using fluorescently labeled anti-Drp1 and anti-E2F5 antibodies. Data are means±SD from 6-8 tumor tissue sections. *p<0.05 as determined by Student's t test.

FIGS. 11A-E. HPV-infection induces tumor suppression and cell death in response to cisplatin. (A-D) Effects of cisplatin on UM-SCC-22A (A) versus UM-SCC-47 (B)-derived xenograft tumors grown in the flanks of SCID mice were measured at days 0 and 14 (n=5-8 mice/group, and *p<0.05). After the measurement of tumor volumes, CerS1-6 mRNAs were measured in extracted tumor tissues from SCID mice treated with cisplatin at day 0 versus day 14 in UM-SCC-22A-(C) or UM-SCC-47-(D)-derived xenograft tumors. CerS1-6 mRNAs were normalized to 28S rRNA. Data are means±SD from three independent experiments. *p<0.05 as determined by Student's t test. (E) Effects of cisplatin on cell death in UM-SCC-22A (HPV−) versus UM-SCC-47 (HPV+) cells were measured by trypan blue exclusion assay. Data are means±SD from three independent experiments. *p<0.05 as determined by Student's t test.

FIGS. 12A-C. Knockdown of autophagy inducer LC3 inhibits cisplatin-mediated cell death in HPV(+) HNSCC cells. (A) Induction of autophagy in response to cisplatin was assessed by detection of LC3B lipidation using Western blotting in HPV(+) UM-SCC-47 cell extracts with anti-LC3B antibody compared to vehicle (veh) treated controls. Actin was used as a loading control. In Western blot panels, images are representative of three independent experiments. Samples shown are from the same representative blot but not in contiguous lanes. (B-C) Effects of siRNA-mediated knockdown of LC3B on cisplatin-mediated cell death were measured and IC₅₀ concentrations were calculated by MTT assays compared to Scr-siRNA-transfected controls. Data are means±SD from three independent experiments. *p<0.05 as determined by Student's t test. Si-RNA-mediated knockdown of LC3B was confirmed using Western blotting with anti-LC3B antibody compared to Scr-siRNA-transfected controls (C). Actin was used as a loading control. In Western blot panels, images are representative of three independent experiments.

FIGS. 13A-B. HPV-E7 enhances mitochondrial ceramide-dependent lethal mitophagy. (A) Effects of siRNA-mediated knockdown of HPVE6/E7 on UM-SCC-47 cell death in response to C₁₈-pyr-cer or vehicle (DMSO) were measured by MTT assay. Scr-siRNA-transfected cells were used as controls. Data are means±SD from three independent experiments. *p<0.05 as determined by Student's t test. (B) Effects of ectopic expression of HPV-E6 versus E7 on HPV(−) UM-SCC-22A cell death in response to C₁₈-pyr-cer or vehicle (DMSO) were measured by MTT assay. Vector-transfected cells were used as controls. Data are means±SD from three independent experiments. *p<0.05 as determined by Student's t test.

FIGS. 14A-C. Analysis of subcellular localization and function of E2F5 in HPV(+) HNSCC cells. (A) Effects of shRNA-mediated knockdown on E2F5 on ATG5, Drp1 and LC3B mRNAs were measured using qRT-PCR in UM-SCC-47 cells compared to Scr-shRNA-transfected controls. Data are means±SD from three independent experiments. *p<0.05 as determined by Student's t test. (B) Subcellular localization of E2F5 was assessed in the presence/absence of C₁₈-pyr-cer by immunofluorescence using fixed confocal micrographs of UM-SCC47 cells stained with DAPI, anti-F-actin and anti-E2F5 antibodies. Images represent at least three independent experiments. (C) Protein abundance of E2F5 in cytoplasm versus nucleus in the presence/absence of cisplatin (0, 1, 2, and 4 h) was detected by Western blotting using cytoplasm- versus nuclei-enriched subcellular fractions of UM-SCC-47 cells with/without anti-E2F5 antibody. Anti-clathrin antibody was used to validate cytoplasmic fractions, whereas anti-lamin B antibody was used to validate nuclear fractions. Western blot images represent at least three independent experiments.

FIG. 15. Graphical summary. Our novel data demonstrate that inhibition of retinoblastoma protein (RB) by the HPV-E7 oncoprotein relieves E2F5, which then associates with Drp1 as a scaffolding protein, resulting in Drp1 activation and/or oligomerization, leading to Drp1-mediated mitochondrial fission, induction of ceramide-dependent lethal mitophagy, and enhanced tumor suppression.

### DESCRIPTION OF ILLUSTRATIVE EMBODIMENTS

Human papillomavirus (HPV) infection is linked to improved survival in response to chemo-radiotherapy for patients with oropharynx head and neck squamous cell carcinoma (HNSCC). However, mechanisms involved in increased HNSCC cell death by HPV signaling in response to therapy are largely unknown. Studies in the present disclosure used molecular, pharmacologic and genetic tools to show that HPV early protein 7 (E7) enhances ceramide-mediated lethal mitophagy in response to chemotherapy-induced cellular stress in HPV-positive HNSCC cells by selectively targeting retinoblastoma protein (RB). Inhibition of RB by HPV-E7 relieves E2F5, which then associates with DRP1, providing a scaffolding platform for Drp1 activation and mitochondrial translocation, leading to mitochondrial fission and increased lethal mitophagy. Ectopic expression of a constitutively active mutant RB, which is not inhibited by HPV-E7, attenuated ceramide-dependent mitophagy and cell death in HPV+HNSCC cells. Moreover, mutation of E2F5 to prevent Drp1 activation inhibited mitophagy in HPV+cells. Activation of Drp1 with E2F5-mimetic peptide for inducing Drp1 mitochondrial localization, enhanced ceramide-mediated mitophagy, and led to tumor suppression in HPV-negative HNSCC—derived xenograft tumors in response to cisplatin in SCID mice.

Thus, the present disclosure provides an E2F5-mimetic polypeptide and its use thereof for the treatment of cancers, particularly head and neck cancer. In particular, the E2F5 polypeptide provided herein is administered in combination with a chemotherapeutic such as cisplatin to provide an improved response to therapy in subjects with head and neck cancer, without pathological HPV infection.

### I. DEFINITIONS

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein in the specification and claims, “a” or “an” may mean one or more. As used herein in the specification and claims, when used in conjunction with the word “comprising”, the words “a” or “an” may mean one or more than one. As used herein, in the specification and claim, “another” or “a further” may mean at least a second or more.

As used herein in the specification and claims, the term “about” is used to indicate that a value includes the inherent variation of error for the device, the method being employed to determine the value, or the variation that exists among the study subjects.

“Treatment” and “treating” refer to administration or application of a therapeutic agent to a subject or performance of a procedure or modality on a subject for the purpose of obtaining a therapeutic benefit of a disease or health-related condition. For example, a treatment may include administration of a T cell therapy.

“Subject” and “patient” refer to either a human or non-human, such as primates, mammals, and vertebrates. In particular embodiments, the subject is a human.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the size of a tumor, a reduction in the invasiveness of a tumor, reduction in the growth rate of the cancer, or prevention of metastasis. Treatment of cancer may also refer to prolonging survival of a subject with cancer.

An “anti-cancer” agent is capable of negatively affecting a cancer cell/tumor in a subject, for example, by promoting killing of cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer.

The phrases “pharmaceutical or pharmacologically acceptable” refers to molecular entities and compositions that do not produce an adverse, allergic, or other untoward reaction when administered to an animal, such as a human, as appropriate. The preparation of a pharmaceutical composition comprising an antibody or additional active ingredient will be known to those of skill in the art in light of the present disclosure. Moreover, for animal (e.g., human) administration, it will be understood that preparations should meet sterility, pyrogenicity, general safety, and purity standards as required by FDA Office of Biological Standards.

As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

A polynucleotide or polynucleotide region (or a polypeptide or polypeptide region) has a certain percentage (for example, 80%, 85%, 90%, or 95%) of “sequence identity” or “homology” to another sequence means that, when aligned, that percentage of bases (or amino acids) are the same in comparing the two sequences. This alignment and the percent homology or sequence identity can be determined using software programs known in the art, for example those described in CURRENT PROTOCOLS IN MOLECULAR BIOLOGY (F. M. Ausubel et al., eds., 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60, expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR.

### II. E2F5 POLYPEPTIDES

Embodiments of the present disclosure concern E2F5 mimetic polypeptides. In particular embodiments, the E2F5 mimetic polypeptide comprises a biologically active fragment of a E2F5 polypeptide. The amino acid sequence of a wild type E2F5 polypeptide (GenBank Accession No. CAB01634.1) is provided below:

| (SEQ ID NO: 13) | |
|---|---|
| 1 | maaaepassg qqapagqgqg qrpppqppqa qapqpppppq lggagggssr hekslglltt |
| 61 | kfvsllqeak dgvldlkaaa dtlavrqkrr iyditnvleg idliekkskn siqwkgvgag |
| 121 | cntkevidrl rylkaeiedl elkereldqq klllqqsikn vmddsinnrf syvthedicn |
| 181 | cfngdtllai qapsgtqlev pipemgqngq kkyqinlksh sgpihvllin kesssskpvv |
| 241 | fpvpppddlt qpssqsltpv tpqkssmatq nlpeqhvser sqalqqtsat dissagsisg |
| 301 | diidelmssd vfpllrlspt paddynfnld dnegvcdlfd vqilny |

In certain preferred aspects, a E2F5 fragment of the embodiments comprises a sequence having at least 80, 85, 90, 91, 92, 93, 94, 95, 96, 97, 98, 99, or 100 percent sequence identity with the dimerization domain of E2F5 provided as SEQ ID NO: 1 (e.g., amino acids 84-177 of GenBank Accession No. CAB01634.1) or with SEQ ID NO: 2. In certain aspects, polypeptide comprises no more that 100, 95, 85, 80, 75, 70, 65, 60, 55, 50, 45, 49, 48, 47, 46, 40, 39, 38, 37, 36, 35, 34, 33, 32, 31, or 30 contiguous amino acids of SEQ ID NO: 13. In particular aspects, the polypeptide comprises, consists of or consists essentially of the amino acid sequence ELDQQKLWLQQSIKNVMDDSINNRFSYVTHED (SEQ ID NO: 2).

An E2F1 polypeptide may be a recombinant polypeptide, synthetic polypeptide, purified polypeptide, immobilized polypeptide, detectably labeled polypeptide, encapsulated polypeptide, or a vector-expressed polypeptide (e.g., a polypeptide encoded by a nucleic acid in a vector comprising a heterologous promoter operably linked to the nucleic acid). In some embodiments, an E2F5 polypeptide may be administered to a subject, such as a human patient, for the treatment of cancer, such as head and neck cancer.

The E2F5 polypeptides that can be used in various embodiments include the amino acid sequences described herein, as well as analogues and derivatives thereof. The analogues and derivatives can include, but are not limited to, additions or substitutions of amino acid residues within the amino acid sequences encoded by a nucleotide sequence, but that result in a silent change, thus producing a functionally equivalent gene product. Amino acid substitutions may be made on the basis of similarity in polarity, charge, solubility, hydrophobicity, hydrophilicity, and/or the amphipathic nature of the residues involved. For example: nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid.

Amino acid substitutions may alternatively be made on the basis of the hydropathic index of amino acids. Each amino acid has been assigned a hydropathic index on the basis of its hydrophobicity and charge characteristics. They are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). The use of the hydropathic amino acid index in conferring interactive biological function on a protein is understood in the art (Kyte and Doolittle, J. Mol. Biol. 157:105-132, 1982). It is known that in certain instances, certain amino acids may be substituted for other amino acids having a similar hydropathic index or score and still retain a similar biological activity. In making changes based upon the hydropathic index, in certain embodiments the substitution of amino acids whose hydropathic indices are within ±2 is included, while in other embodiments amino acid substitutions that are within ±1 are included, and in yet other embodiments amino acid substitutions within ±0.5 are included.

Amino acid substitutions may alternatively be made on the basis of hydrophilicity, particularly where the biologically functional protein or polypeptide thereby created is intended for use in immunological embodiments. In certain embodiments, the greatest local average hydrophilicity of a protein, as governed by the hydrophilicity of its adjacent amino acids, correlates with its immunogenicity and antigenicity, i.e., with a biological property of the protein. The following hydrophilicity values have been assigned to these amino acid residues: arginine (+3.0); lysine (+3.0); aspartate (+3.0 ±1); glutamate (+3.0 ±1); serine (+0.3); asparagine (+0.2); glutamine (+0.2); glycine (0); threonine (−0.4); proline (−0.5 ±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In making changes based upon similar hydrophilicity values, in certain embodiments the substitution of amino acids whose hydrophilicity values are within ±2 is included, in certain embodiments those that are within ±1 are included, and in certain embodiments those within ±0.5 are included. One may also identify epitopes from primary amino acid sequences on the basis of hydrophilicity.

Substitutional variants typically contain the exchange of one amino acid for another at one or more sites within the protein, and may be designed to modulate one or more properties of the polypeptide, with or without the loss of other functions or properties. Substitutions may be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Alternatively, substitutions may be non-conservative such that a function or activity of the polypeptide is affected. Non-conservative changes typically involve substituting a residue with one that is chemically dissimilar, such as a polar or charged amino acid for a nonpolar or uncharged amino acid, and vice versa.

A. Cell Penetration Sequence

The E2F5 polypeptide of the present disclosure may comprise or be coupled to a cell importation peptide or a cellular internalization transporter (e.g., via a peptide bond, linker, or cleavable linker). As used herein the term “cell penetrating peptide” refers to segments of polypeptide sequence that allow or promote a polypeptide to cross the cell membrane, such as the plasma membrane of a eukaryotic cell. Examples of cell importation signals include, but are not limited to, polyarginine sequences (e.g., RRRRRRRR (SEQ ID NO: 7), segments derived from HIV Tat (e.g., GRKKRRQRRRPPQ, SEQ ID NO:8; or RKKRRQRRR, SEQ ID NO: 9), herpes virus VP22, the Drosophila Antennapedia homeobox gene product (RQPKIWFPNRRKPWKK; SEQ ID NO:10), protegrin I, Penetratin (RQIKIWFQNRRMKWKK; SEQ ID NO:11), Antp-3A (Antp mutant), Buforin II Transportan, MAP (model amphipathic peptide), K-FGF, Ku70, Prion, pVEC, Pep-I, SynBl, Pep-7, HN-1, KALA, R11, K11, or melittin (GIGAVLKVLTTGLPALISWIKRKRQQ; SEQ ID NO:12). Poly-R sequences may vary in length, e.g., from 3, 4, 5, 6, 7, 8, 9, 10, 11, 12, 13, 14, or 15 R amino acids in length.

Cell importation signals for use herein may be covalently conjugated (e.g., chemically fused or attached, expressed as a fusion construct, etc.) with an E2F5 polypeptide to promote transport of the E2F5 polypeptide across a cell membrane. Cell importation signals that may be used include, e.g., peptides (e.g., cell penetration peptides), polypeptides, hormones, growth factors, cytokines, aptamers or avimers. Furthermore, a cell importation signal may mediate non-specific cell internalization or may be a cell targeting moiety that is internalized in a subpopulation of targeted cells.

B. Cell-Targeting Moiety

In some embodiments, an E2F5 polypeptide may be expressed as a fusion protein or chemically attached to a cell targeting moiety to selectively target the construct containing the E2F5 polypeptide to a particular subset of cells such as, e.g., cancerous cells, tumor cells, endothelial cells. For example, in some embodiments, the cell targeting moiety is an antibody. In general the term antibody includes, but is not limited to, polyclonal antibodies, monoclonal antibodies, single chain antibodies, humanized antibodies, minibodies, dibodies, tribodies as well as antibody fragments, such as Fab′, Fab, F(ab′)2, single domain antibodies and any mixture thereof. In some cases it is preferred that the cell targeting moiety is a single chain antibody (scFv). In a related embodiment, the cell targeting domain may be an avimer polypeptide. Therefore, in certain cases the cell targeting constructs of the present disclosure are fusion proteins comprising an E2F5 polypeptide and a scFv or an avimer.

In certain aspects of the present disclosure, a cell targeting moiety may be a growth factor. For example, transforming growth factor, epidermal growth factor, insulin-like growth factor, fibroblast growth factor, B lymphocyte stimulator (BLyS), heregulin, platelet-derived growth factor, vascular endothelial growth factor (VEGF), or hypoxia inducible factor may be used as a cell targeting moiety according to the invention. These growth factors enable the targeting of constructs to cells that express the cognate growth factor receptors.

In further aspects of the present disclosure, a cell targeting moiety may be a hormone. Some examples of hormones for use in the invention include, but are not limited to, human chorionic gonadotropin, gonadotropin releasing hormone, an androgen, an estrogen, thyroid-stimulating hormone, follicle-stimulating hormone, luteinizing hormone, prolactin, growth hormone, adrenocorticotropic hormone, antidiuretic hormone, oxytocin, thyrotropin-releasing hormone, growth hormone releasing hormone, corticotropin-releasing hormone, somatostatin, dopamine, melatonin, thyroxine, calcitonin, parathyroid hormone, glucocorticoids, mineralocorticoids, adrenaline, noradrenaline, progesterone, insulin, glucagon, amylin, erythropoitin, calcitriol, calciferol, atrial-natriuretic peptide, gastrin, secretin, cholecystokinin, neuropeptide Y, ghrelin, PYY3-36, insulin-like growth factor-1, leptin, thrombopoietin, angiotensinogen, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, or IL-36. Targeting constructs that comprise a hormone may enable methods of targeting cell populations that comprise extracelluar receptors for the indicated hormone.

In certain aspects, a cell targeting moiety of the present disclosure may be a cancer cell-targeting moiety. It is well known that certain types of cancer cells aberrantly express surface molecules that are unique as compared to surrounding tissue. Thus, cell targeting moieties that bind to these surface molecules may enable the targeted delivery of E2F5 peptides specifically to the cancers cells. For example, a cell targeting moiety may bind to and be internalized by a lung, breast, brain, prostate, spleen, pancreatic, cervical, ovarian, head and neck, esophageal, liver, skin, kidney, leukemia, bone, testicular, colon, or bladder cancer cell. The skilled artisan will understand that the effectiveness of a cancer cell-targeted E2F5 polypeptide may, in some cases, be contingent upon the expression or expression level of a particular cancer marker on the cancer cell. Thus, in certain aspects, there are provided methods for treating a cancer with a targeted E2F5 polypeptide comprising determining whether (or to what extent) the cancer cell expresses a particular cell surface marker and administering targeted E2F5 polypeptide therapy (or another anticancer therapy) to the cancer cells depending on the expression level of a marker gene or polypeptide.

C. Linkers/Coupling Agents

In some embodiments, an E2F5 polypeptide of the present disclosure may be chemically attached to another group such as, e.g., a cell targeting moiety. If desired, the compound of interest may be joined via a biologically-releasable bond, such as a selectively-cleavable linker or amino acid sequence. For example, peptide linkers that include a cleavage site for an enzyme preferentially located or active within a tumor environment are contemplated. Exemplary forms of such peptide linkers are those that are cleaved by urokinase, plasmin, thrombin, Factor IXa, Factor Xa, or a metallaproteinase, such as collagenase, gelatinase, or stromelysin.

Additionally, while numerous types of disulfide-bond containing linkers are known which can successfully be employed to conjugate moieties, certain linkers will generally be preferred over other linkers, based on differing pharmacologic characteristics and capabilities. For example, linkers that contain a disulfide bond that is sterically “hindered” may be preferred, due to their greater stability in vivo, thus preventing release of the moiety prior to binding at the site of action.

Additionally, any other linking/coupling agents and/or mechanisms known to those of skill in the art can be attached to a peptide of the present invention, such as, for example, amide linkages, ester linkages, thioester linkages, ether linkages, thioether linkages, phosphoester linkages, phosphoramide linkages, anhydride linkages, disulfide linkages, ionic and hydrophobic interactions, or combinations thereof.

Cross-linking reagents are used to form molecular bridges that tie together functional groups of two different molecules, e.g., a stablizing and coagulating agent. However, it is contemplated that dimers or multimers of the same analog can be made or that heteromeric complexes comprised of different analogs can be created. To link two different compounds in a step-wise manner, hetero-bifunctional cross-linkers can be used that eliminate unwanted homopolymer formation.

### III. METHODS OF TREATMENT

Provided herein are methods for treating or delaying progression of cancer in an individual comprising administering to the individual an effective amount an E2F5 polypeptide provided herein. Examples of cancers contemplated for treatment include lung cancer, head and neck cancer, breast cancer, pancreatic cancer, prostate cancer, renal cancer, bone cancer, testicular cancer, cervical cancer, gastrointestinal cancer, lymphomas, pre-neoplastic lesions in the lung, colon cancer, melanoma, and bladder cancer. In particular aspects, the cancer is head and neck cancer.

The E2F5 polypeptide may be administered intravenously, intramuscularly, subcutaneously, topically, orally, transdermally, intraperitoneally, intraorbitally, by implantation, by inhalation, intrathecally, intraventricularly, or intranasally. The appropriate dosage of the E2F5 polypeptide may be determined based on the type of disease to be treated, severity and course of the disease, the clinical condition of the individual, the individual's clinical history and response to the treatment, and the discretion of the attending physician.

Intratumoral injection, or injection into the tumor vasculature is specifically contemplated for discrete, solid, accessible tumors. Local, regional or systemic administration also may be appropriate. For tumors of >4 cm, the volume to be administered will be about 4-10 ml (in particular 10 ml), while for tumors of <4 cm, a volume of about 1-3 ml will be used (in particular 3 ml). Multiple injections delivered as single dose comprise about 0.1 to about 0.5 ml volumes.

A. Pharmaceutical Compositions

Also provided herein are pharmaceutical compositions and formulations comprising the E2F5 polypeptide and a pharmaceutically acceptable carrier.

Pharmaceutical compositions and formulations as described herein can be prepared by mixing the active ingredients (such as an antibody or a polypeptide) having the desired degree of purity with one or more optional pharmaceutically acceptable carriers (Remington's Pharmaceutical Sciences 22^{nd} edition, 2012), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn- protein complexes); and/or non-ionic surfactants such as polyethylene glycol (PEG).

B. Combination Therapies

In certain embodiments, the compositions and methods of the present embodiments involve an E2F5 polypeptide in combination with at least one additional therapy. The additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, or a combination of the foregoing. The additional therapy may be in the form of adjuvant or neoadjuvant therapy. In particular aspects, the at least one additional therapy is a chemotherapy (e.g., cisplatin) or a ceramide analogue drug.

In some embodiments, the additional therapy is the administration of small molecule enzymatic inhibitor or anti-metastatic agent. In some embodiments, the additional therapy is the administration of side- effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy is therapy targeting PBK/AKT/mTOR pathway, HSP90 inhibitor, tubulin inhibitor, apoptosis inhibitor, and/or chemopreventative agent. The additional therapy may be one or more of the chemotherapeutic agents known in the art.

An E2F5 polypeptide composition may be administered before, during, after, or in various combinations relative to an additional cancer therapy. The administrations may be in intervals ranging from concurrently to minutes to days to weeks. In embodiments where the E2F5 polypeptide composition is provided to a patient separately from an additional therapeutic agent, one would generally ensure that a significant period of time did not expire between the time of each delivery, such that the two compounds would still be able to exert an advantageously combined effect on the patient. In such instances, it is contemplated that one may provide a patient with the antibody therapy and the anti-cancer therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

Various combinations may be employed. For the example below an E2F5 polypeptide therapy is “A” and an anti-cancer therapy is “B”:

| | | | | | |
|---|---|---|---|---|---|
| A/B/A | B/A/B | B/B/A | A/A/B | A/B/B | B/A/A |
| A/B/B/B | B/A/B/B | B/B/B/A | B/B/A/B | A/A/B/B | A/B/A/B |
| A/B/B/A | B/B/A/A | B/A/B/A | B/A/A/B | A/A/A/B | B/A/A/A |
| A/B/A/A | A/A/B/A | | | | |

Administration of any compound or therapy of the present embodiments to a patient will follow general protocols for the administration of such compounds, taking into account the toxicity, if any, of the agents. Therefore, in some embodiments there is a step of monitoring toxicity that is attributable to combination therapy.

1. Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammall and calicheamicin omegaIl); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine,plicomycin, gemcitabien, navelbine, farnesyl-protein tansferase inhibitors, transplatinum, and pharmaceutically acceptable salts, acids, or derivatives of any of the above

2. Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

3. Immunotherapy

The skilled artisan will understand that additional immunotherapies may be used in combination or in conjunction with methods of the embodiments. In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T cells and NK cells

Antibody-drug conjugates have emerged as a breakthrough approach to the development of cancer therapeutics. Cancer is one of the leading causes of deaths in the world. Antibody-drug conjugates (ADCs) comprise monoclonal antibodies (MAbs) that are covalently linked to cell-killing drugs. This approach combines the high specificity of MAbs against their antigen targets with highly potent cytotoxic drugs, resulting in “armed” MAbs that deliver the payload (drug) to tumor cells with enriched levels of the antigen. Targeted delivery of the drug also minimizes its exposure in normal tissues, resulting in decreased toxicity and improved therapeutic index. The approval of two ADC drugs, ADCETRIS® (brentuximab vedotin) in 2011 and KADCYLA® (trastuzumab emtansine or T-DM1) in 2013 by FDA validated the approach. There are currently more than 30 ADC drug candidates in various stages of clinical trials for cancer treatment (Leal et al., 2014). As antibody engineering and linker-payload optimization are becoming more and more mature, the discovery and development of new ADCs are increasingly dependent on the identification and validation of new targets that are suitable to this approach and the generation of targeting MAbs. Two criteria for ADC targets are upregulated/high levels of expression in tumor cells and robust internalization.

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, 1998; Christodoulides et al., 1998); cytokine therapy, e.g., interferons α, β, and γ, IL-1, GM-CSF, and TNF (Bukowski et al., 1998; Davidson et al., 1998; Hellstrand et al., 1998); gene therapy, e.g., TNF, IL-1, IL-2, and p53 (Qin et al., 1998; Austin-Ward and Villaseca, 1998; U.S. Pat. Nos. 5,830,880 and 5,846,945); and monoclonal antibodies, e.g., anti-CD20, anti-ganglioside GM2, and anti-p185 (Hollander, 2012; Hanibuchi et al., 1998; U.S. Pat. No. 5,824,311). It is contemplated that one or more anti-cancer therapies may be employed with the antibody therapies described herein.

In some embodiments, the immunotherapy may be an immune checkpoint inhibitor. Immune checkpoints either turn up a signal (e.g., co-stimulatory molecules) or turn down a signal. Inhibitory immune checkpoints that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, are antibodies, such as human antibodies (e.g., International Patent Publication WO2015016718; Pardoll, Nat Rev Cancer, 12(4): 252-64, 2012; both incorporated herein by reference). Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present invention. For example it is known that lambrolizumab is also known under the alternative and equivalent names MK-3475 and pembrolizumab.

In some embodiments, the PD-1 binding antagonist is a molecule that inhibits the binding of PD-1 to its ligand binding partners. In a specific aspect, the PD-1 ligand binding partners are PDL1 and/or PDL2. In another embodiment, a PDL1 binding antagonist is a molecule that inhibits the binding of PDL1 to its binding partners. In a specific aspect, PDL1 binding partners are PD-1 and/or B7-1. In another embodiment, the PDL2 binding antagonist is a molecule that inhibits the binding of PDL2 to its binding partners. In a specific aspect, a PDL2 binding partner is PD-1. The antagonist may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 axis antagonists for use in the methods provided herein are known in the art such as described in U.S. Patent Application No. US20140294898, US2014022021, and US20110008369, all incorporated herein by reference.

In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PD-1 binding antagonist is AMP- 224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PDL2-Fc fusion soluble receptor described in WO2010/027827 and WO2011/066342.

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to CD80 and CD86, also called B7-1 and B7-2 respectively, on antigen-presenting cells. CTLA4 transmits an inhibitory signal to T cells, whereas CD28 transmits a stimulatory signal. Intracellular CTLA4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al. (1998) Proc Natl Acad Sci USA 95(17): 10067-10071; Camacho et al. (2004) J Clin Oncology 22(145): Abstract No. 2505 (antibody CP-675206); and Mokyr et al. (1998) Cancer Res 58:5301-5304 can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001014424, WO2000037504, and U.S. Pat. No. 8,017,114; all incorporated herein by reference.

An exemplary anti-CTLA-4 antibody is ipilimumab (also known as 10D1, MDX- 010, MDX- 101, and Yervoy®) or antigen binding fragments and variants thereof (see, e.g., WOO 1/14424). In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2 and CDR3 domains of the VL region of ipilimumab. In another embodiment, the antibody competes for binding with and/or binds to the same epitope on CTLA-4 as the above- mentioned antibodies. In another embodiment, the antibody has at least about 90% variable region amino acid sequence identity with the above-mentioned antibodies (e.g., at least about 90%, 95%, or 99% variable region identity with ipilimumab).

Other molecules for modulating CTLA-4 include CTLA-4 ligands and receptors such as described in U.S. Pat. Nos. 5,844,905, 5,885,796 and International Patent Application Nos. WO1995001994 and WO1998042752; all incorporated herein by reference, and immunoadhesins such as described in U.S. Pat. No. 8,329,867, incorporated herein by reference.

4. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

5. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### IV. ARTICLES OF MANUFACTURE OR KITS

An article of manufacture or a kit is provided comprising E2F5 polypeptides is also provided herein. The article of manufacture or kit can further comprise a package insert comprising instructions for using the E2F5 polypeptides to treat or delay progression of cancer in an individual or to enhance immune function of an individual having cancer. Any of the E2F5 peptides described herein may be included in the article of manufacture or kits. Suitable containers include, for example, bottles, vials, bags and syringes. The container may be formed from a variety of materials such as glass, plastic (such as polyvinyl chloride or polyolefin), or metal alloy (such as stainless steel or hastelloy). In some embodiments, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some embodiments, the article of manufacture further includes one or more of another agent (e.g., a chemotherapeutic agent, and anti-neoplastic agent). Suitable containers for the one or more agent include, for example, bottles, vials, bags and syringes.

### V. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

Example 1—Head and Neck Squamous Cell Carcinoma (HNSCC) Studies

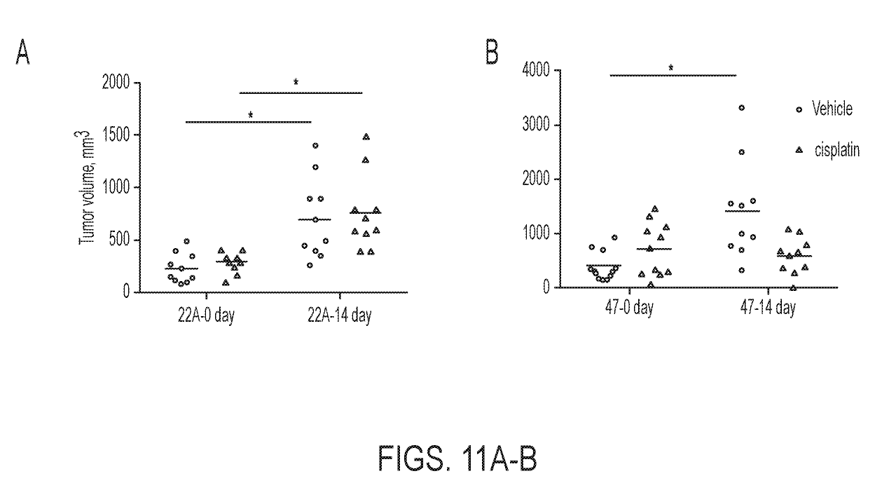

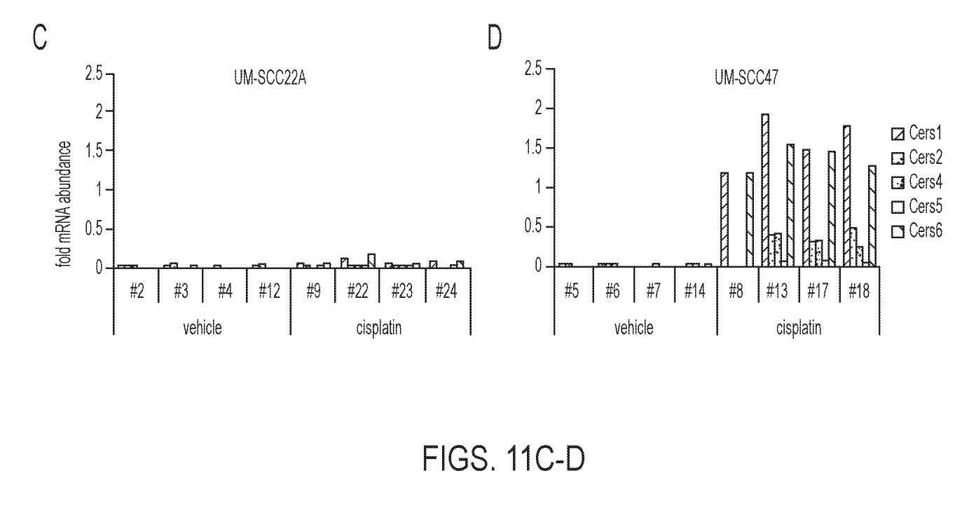

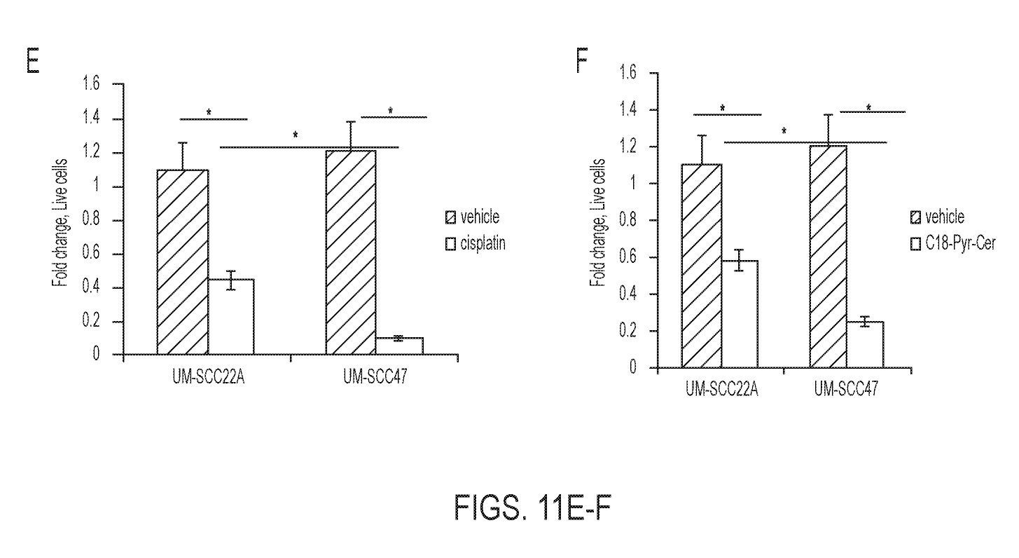

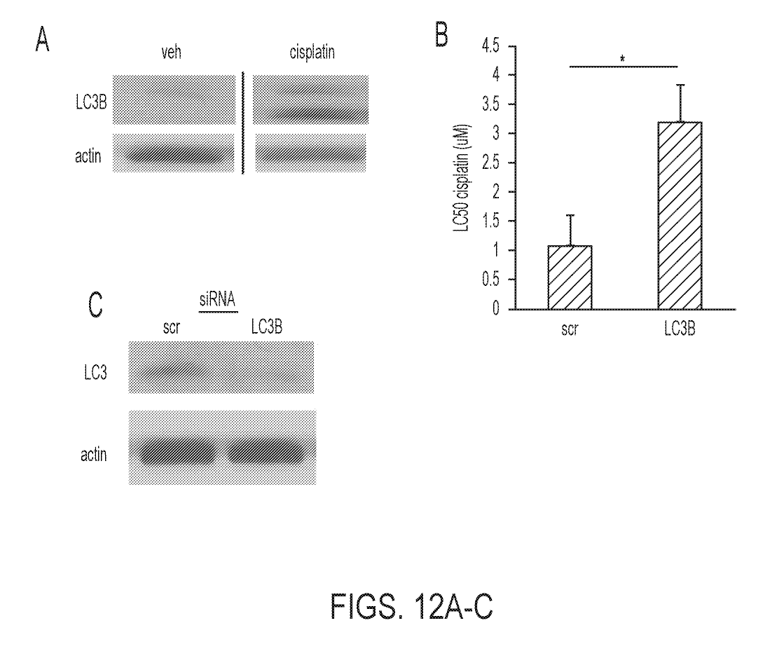

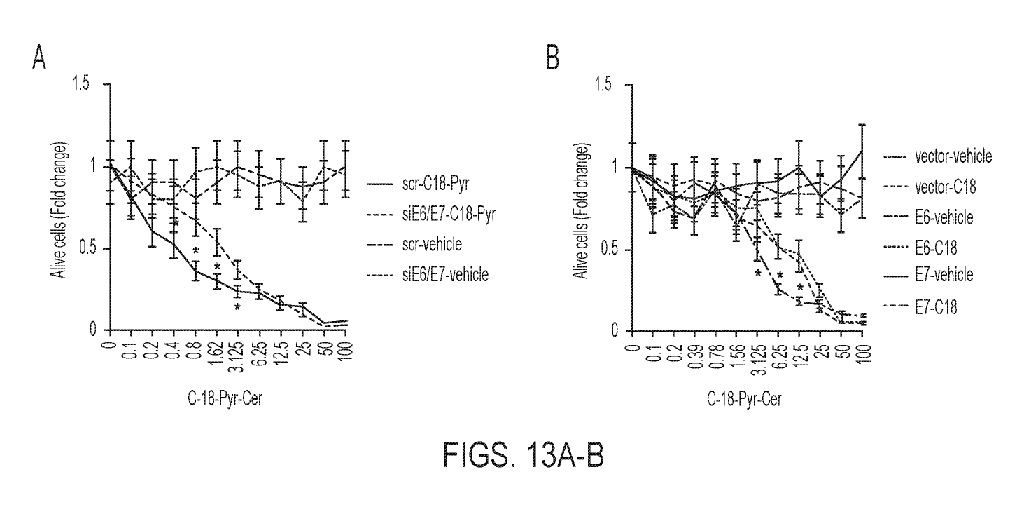

To determine whether HPV(+) compared to HPV(−) HNSCC cells are more sensitive to stress-mediated growth inhibition in response to chemotherapeutic drugs, it was first established HPV(+) UM-SCC-47- and HPV(−) UM-SCC-22A-derived xenograft tumors in SCID mice. After the xenograft-derived tumors were ˜75-100 mm³, the mice were treated with cisplatin (3.5 mg/kg) for two weeks, which is below its maximum tolerated doses (van Moorsel et al, 1999). Then, tumor volumes were measured at days 0 and 14 of treatment. The data suggest that the growth of HPV(+) UM-SCC-47 derived HNSCC xenografts was inhibited in response to cisplatin (˜75% inhibition), whereas there was no detectable tumor suppression when HPV(−) UM-SCC-22A-derived xenografts were treated with this drug in SCID mice (FIGS. 11A-B). After tumor growth measurements in the mice were taken, the tumors were surgically removed and the effects of cisplatin on CerS1-6 mRNA were measured by qRT-PCR. Cisplatin induced CerS1 and CerS6 mRNA in HPV(+) but not in HPV(−) xenograft-derived tumors (FIGS. 11C-D). These data were also consistent when the effects of cisplatin on the growth of these cells in culture were measured, in which HPV(+) UM-SCC-47 cells exhibited ˜3-fold sensitivity for cisplatin-induced growth inhibition/cell death compared to HPV(−) UM-SCC-22A cells in culture (FIG. 11E). Similar data were obtained using C₁₈-pyridinium-ceramide (C₁₈-pyr-cer), a ceramide analogue drug: HPV(+) UM-SCC-47 cells exhibited ˜3-fold sensitivity for C₁₈-pyr-cer-induced growth inhibition/cell death compared to HPV(−) UM-SCC-22A cells in culture (FIG. 11F). The inhibitory concentrations that inhibited growth by 50% (IC50) values of cisplatin and C₁₈-pyr-cer were 8.0 versus 28 μM, and 1.6 versus 5.0 μM, for UM-SCC-47 versus UM-SCC-22A cells, respectively, at 48 h. Thus, these data suggest that UM-SCC-47 (HPV+) cells exhibit more sensitivity for cisplatin- and C₁₈-pyr-cer-mediated growth inhibition and/or cell death than HPV(−) UM-SCC-22A cells/tumors in culture and/or in mice, recapitulating clinical data observed in HPV(+) versus HPV(−) HNSCC patients.

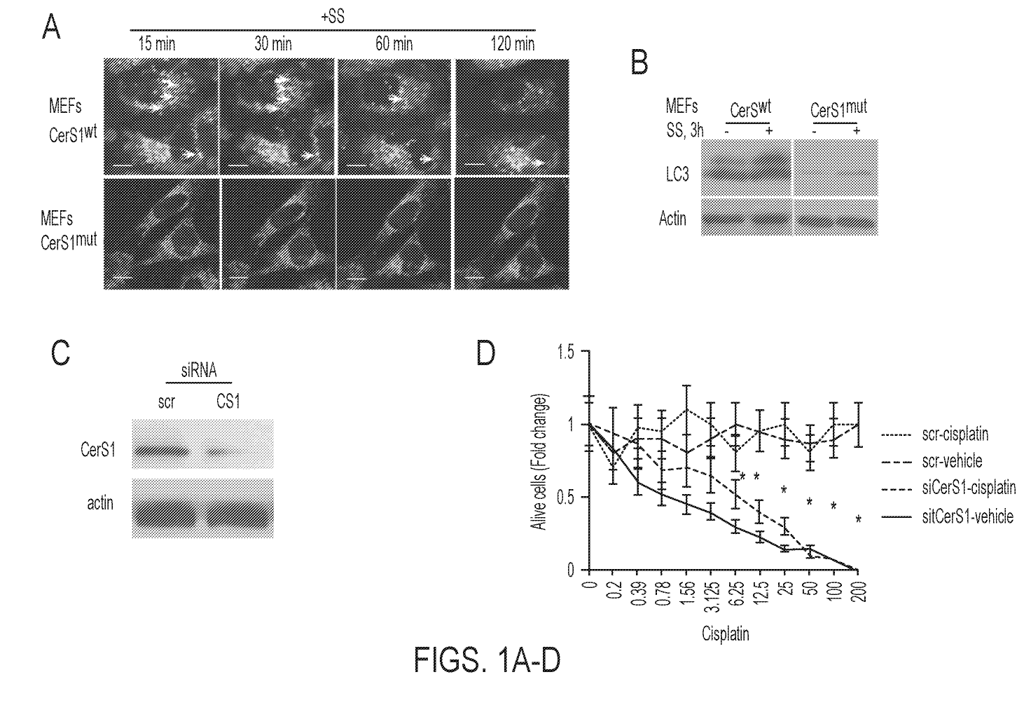

HPV-E7 signaling induces cell death by CerS1/ceramide-dependent mitophagy. Endogenous ceramide (C₁₈-ceramide), which induces lethal mitophagy, is generated by ceramide synthase 1 (CerS1) (Pewzner-Jung et al, 2006; Koybasi et al, 2004; Venkataraman et al, 2002). This is first confirmed by treatment of mouse embryonic fibroblasts (MEFs) isolated from wild type (WT) and CerS1-toppler mutant mice (CerS1^{top/top}), which encodes for inactive enzyme for defective C₁₈-ceramide generation (Spassieva et al, 2016; Zhao et al, 2011) by sodium selenite, a known inducer of mitophagy (Sentelle et al, 2012). Sodium selenite markedly induced mitophagy in WT-MEFs, but not in CerS1^{top/top}-MEFs, as detected by the co-localization of lysosomes (lysotracker green, LTG) and mitochondria (mitotracker red, MTR) using live cell imaging/confocal microscopy or lipidation of LC3B using Western blotting (FIG. 1A-B). Reconstitution of WT-CerS1 expression, and not catalytically inactive mutant CerS1 (H183A) (Sentelle et al, 2012), restored sodium selenite-mediated mitophagy in CerS1^{top/top} MEFs (FIG. 1A-B). Then, to determine whether CerS1/ceramide-signaling plays any roles in cisplatin-mediated growth inhibition in HPV(+) HNSCC cells, the effects of shRNA-mediated knockdown of CerS1 on the growth of UM-SCC-47 cells were measured in response to cisplatin using MTT assay. Knockdown of CerS1, ˜70% downregulation protein compared to scrambled (Scr) shRNA-transfected cells, measured by Western blotting, reduced cisplatin-mediated growth inhibition (˜4-fold) compared to controls (FIG. 1C-D). Thus, these data suggest that CerS1/ceramide-signaling plays a key role, at least in part, in cisplatin-induced growth inhibition in HPV(+) UM-SCC-47 cells.

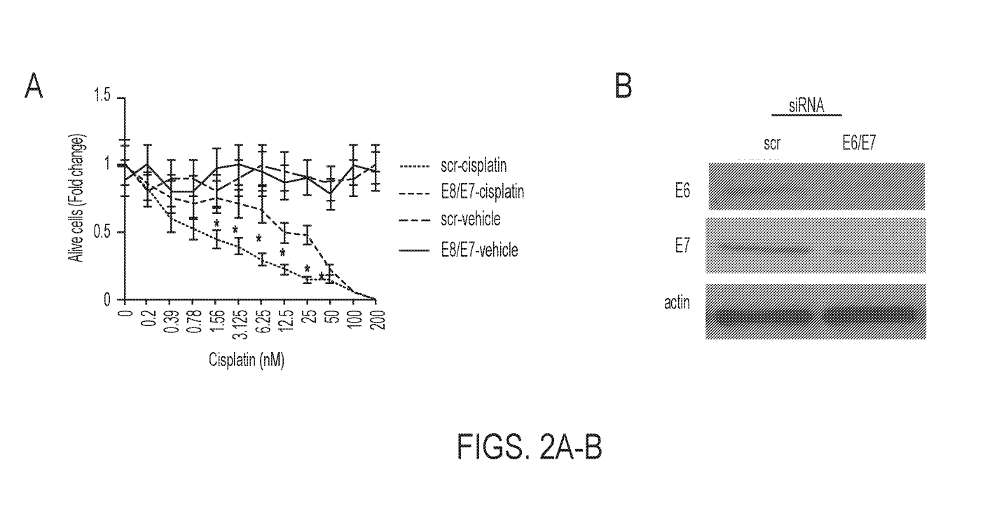

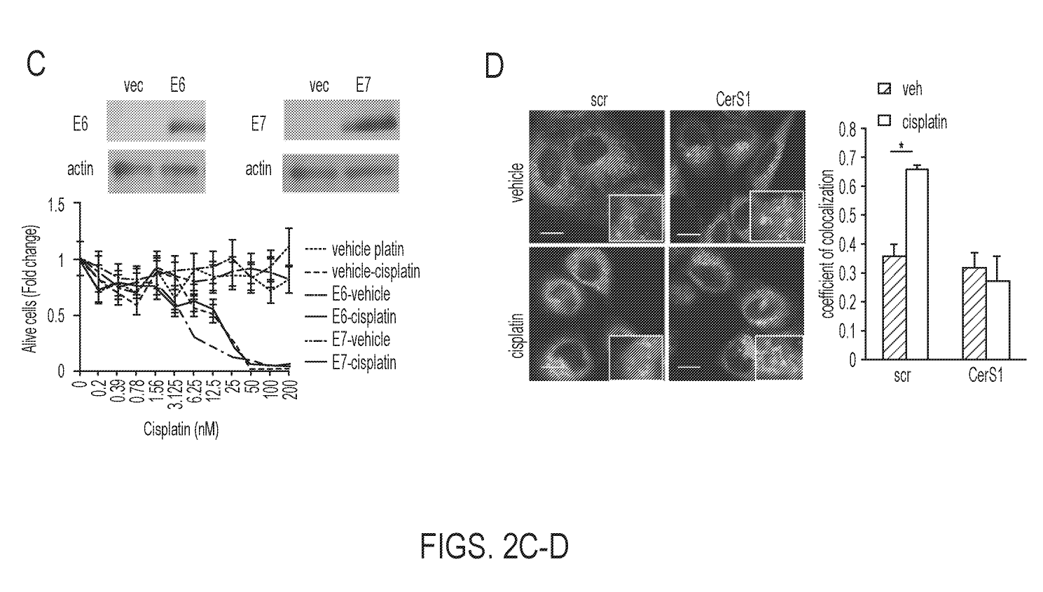

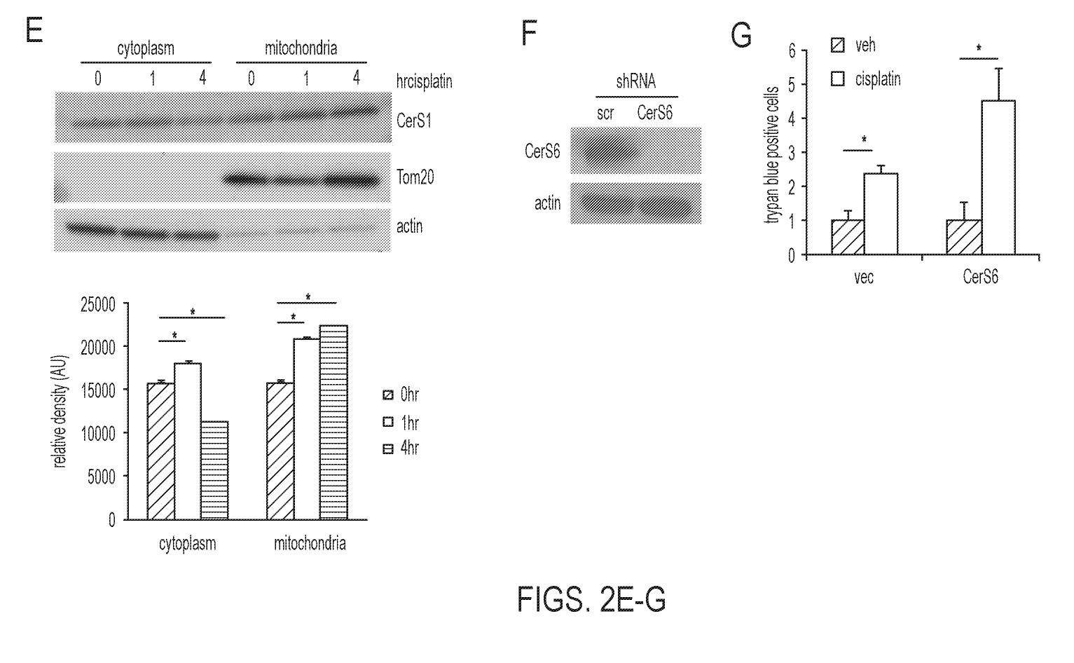

To determine the roles of HPV16-E6 and E7 proteins in HNSCC growth inhibition in response to cisplatin, the effects of siRNA-mediated knockdown of these proteins on the growth of UM-SCC-47 cells were measured in response to cisplatin compared to scrambled (Scr)-siRNA-transfected controls. Knockdown of E6 and E7 proteins, confirmed by Western blotting resulted in ˜8-fold resistance to cisplatin-mediated growth inhibition in UM-SCC-47 cells compared to controls (FIG. 2A-B). To determine if HPV-E6 versus E7 plays distinct roles in the regulation of growth inhibition in response to chemotherapy, these proteins were ectopically expressed selectively (confirmed by Western blotting) in HPV(−) UM-SCC-22A cells, and their effects on growth inhibition or mitophagy were measured in response to cisplatin. Ectopic expression of HPV-E7, but not E6, sensitized UM-SCC-22A cells to cisplatin-mediated cell death (FIG. 2C). ShRNA-mediated knockdown of CerS1 completely abrogated co-localization of lysotracker green (LTG) and mitotracker red (MTR) in response to cisplatin (FIG. 2D). These data were further confirmed by increased accumulation of CerS1 in mitochondria in response to cisplatin (1 and 4 h) compared to controls (0 h), using Western blotting in cytoplasm versus mitochondria enriched cellular fractions (FIG. 2E). As an additional control, the effects of silencing CerS6, which mainly generates C₁₆-ceramide, on cisplatin-induced cell death in UM-SCC-47 cells were also measured. The data showed that shRNA-mediated knockdown of CerS6, confirmed by Western blotting, had no inhibitory effect on cell death in response to cisplatin (FIG. 2F-G). Thus, these data suggest that HPV-E7 enhances CerS1/ceramide-dependent lethal mitophagy in response to cisplatin, regulated by mitochondrial localization/accumulation of CerS 1 /ceramide.

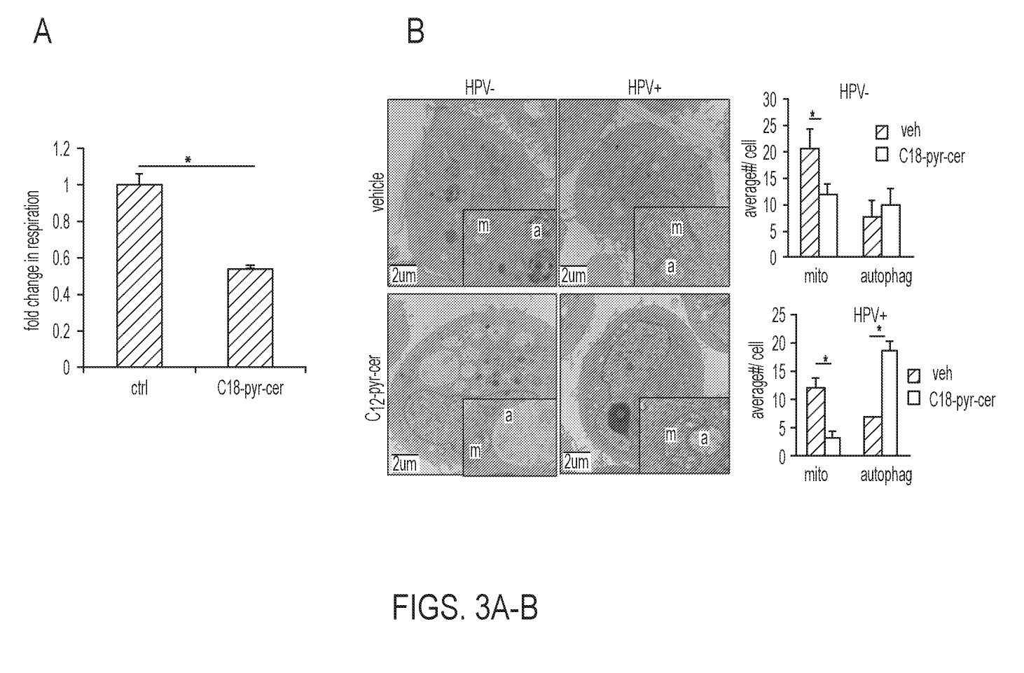

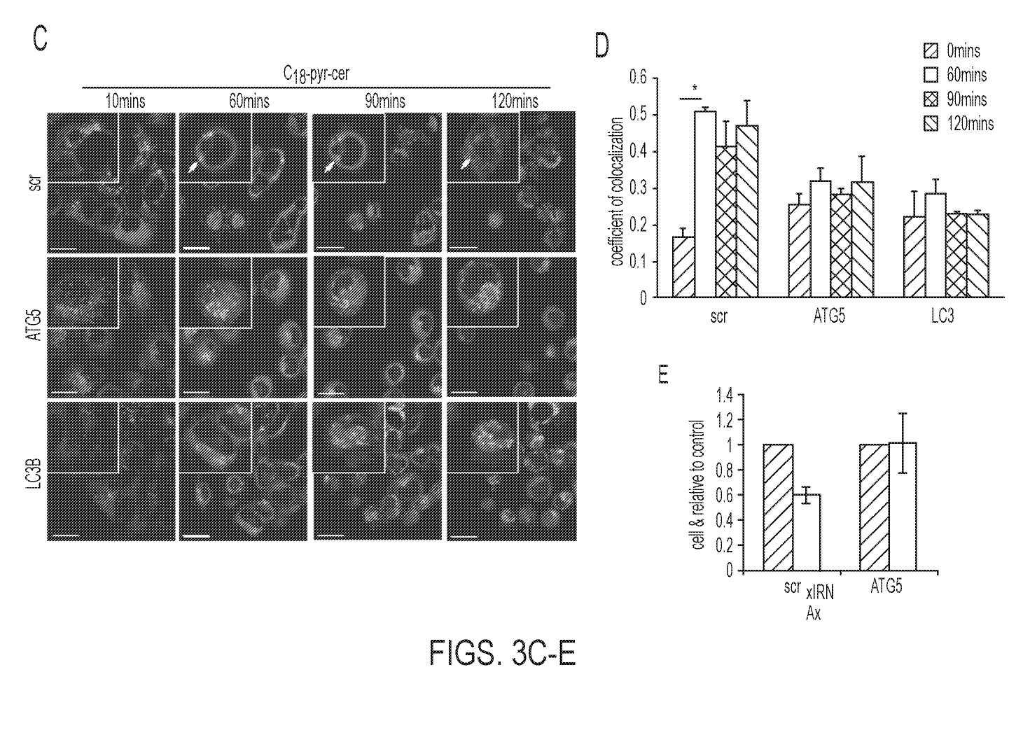

Lethal mitophagy is mediated by mitochondria-targeted C₁₈-ceramide via HPV-E7 signaling. To determine whether ceramide accumulation in mitochondria is sufficient to replicate the cellular response to cisplatin, HPV(+) UM-SCC-47 cells were treated with C₁₈-pyr-cer. A pyridinium ring conjugated to the sphingosine backbone targets this ceramide analogue to mitochondria (Senkal et al, 2006). C₁₈-pyr-cer increased mitophagy detected by decreased oxygen consumption rate, measured using the Sea Horse compared to vehicle-treated controls (FIG. 3A). Additionally, using transmission electron microscopy (TEM), a decreased number mitochondria and an increased number of autophagosomes were detected (suggesting increased mitophagy) in response to C₁₈-pyr-cer in HPV(+) UM-SCC-47 compared to HPV(−) UM-SCC-22A cells (FIG. 3B). Induction of mitophagy by C₁₈-pyr-cer was also measured by live cell imaging using immunofluorescence to detect time-dependent degradation of mitochondria by autophagosomes (FIG. 3C-D). SiRNA-mediated knockdown of LC3B or ATGS prevented C₁₈-pyr-cer-mediated mitophagy and cell death compared to Scr-siRNA-transfected controls (FIG. 3 C-E). Increased LC3B lipidation in response to cisplatin, a marker of autophagy induction, and successful knockdown of LC3B using shRNA were confirmed by Western blot analyses (FIGS. 12A-C). Thus, these data demonstrate that induction of mitochondrial accumulation of C₁₈-pyr-cer enhances mitophagy-dependent cell death in HPV(+) compared to HPV(−) HNSCC cells. It should be noted that C₁₈-pyr-cer induces lethal mitophagy in HPV(−) HNSCC (UM-SCC-22A) cells when used at higher concentrations or longer treatment time points (5-10 μM, 24-48 h), when IC50 is reached (FIG. 11F), as described previously (Sentelle et al, 2012).

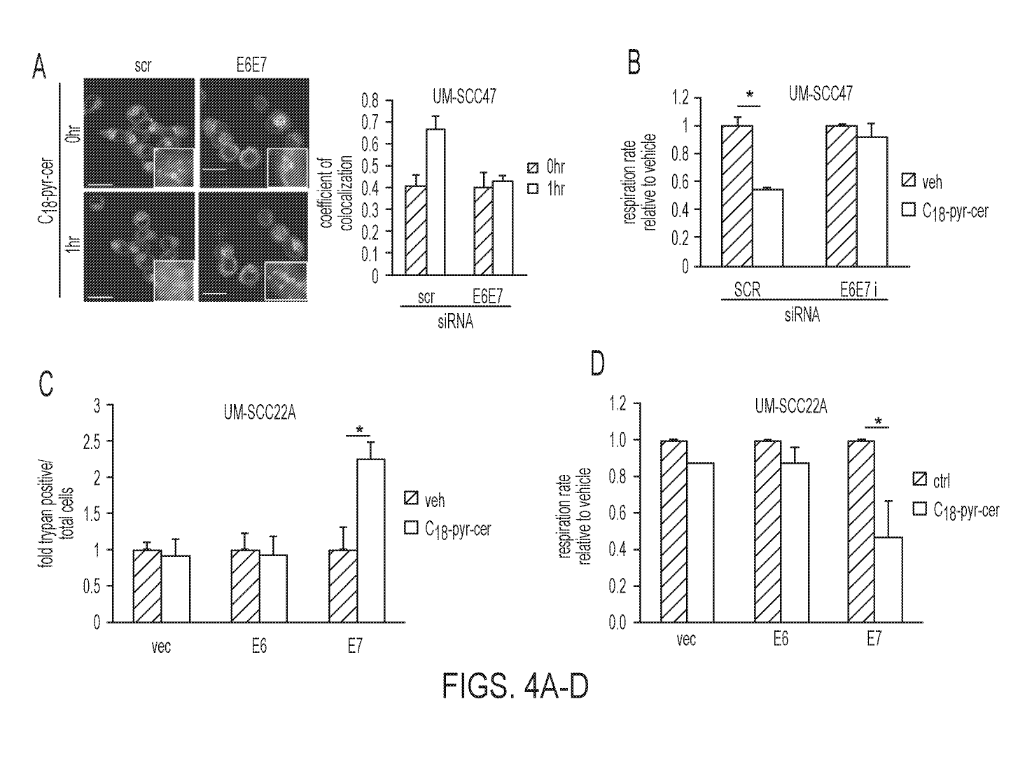

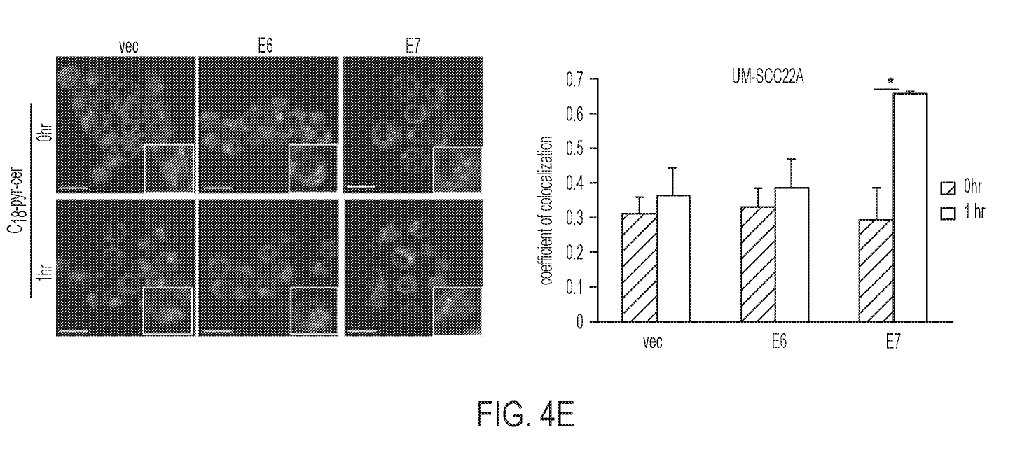

Inhibition of Rb by HPV-E7 targets HNSCC mitochondria for ceramide-dependent mitophagy. To examine a possible role for HPV-E6/E7 oncoproteins in the enhanced response of HPV-positive HNSCC, HPV-E6/E7 in HPV-positive cells were knocked down and the response to treatment with C₁₈-pyr-cer was examined. It was found that knockdown of E6/E7 resulted in resistance to C₁₈-pyr-cer-induced cell death compared to Scr-siRNA-transfected controls (Fig. EV3, A). Moreover, siRNA-mediated knockdown of E6/E7 attenuated C₁₈-pyr-cer-induced mitophagy, measured by change in oxygen consumption rate (OCR), and co-localization of LTG and MTR (FIG. 4A-B). However, ectopic expression of E7, but not E6, resulted in increased cell death in response to C₁₈-pyr-cer in HPV(−) UM-SCC-22A cells, and enhanced mitophagy, measured by oxygen consumption rate (OCR) using the Sea Horse, or co-localization of LTG and MTR, in response to C₁₈-pyr-cer (Fig. EV3, B, and FIG. 4C-E). Taken together, these data suggest that HPV-E7 plays a key role in sensitizing HNSCC cells to treatment with either cisplatin or C₁₈-pyr-cer.

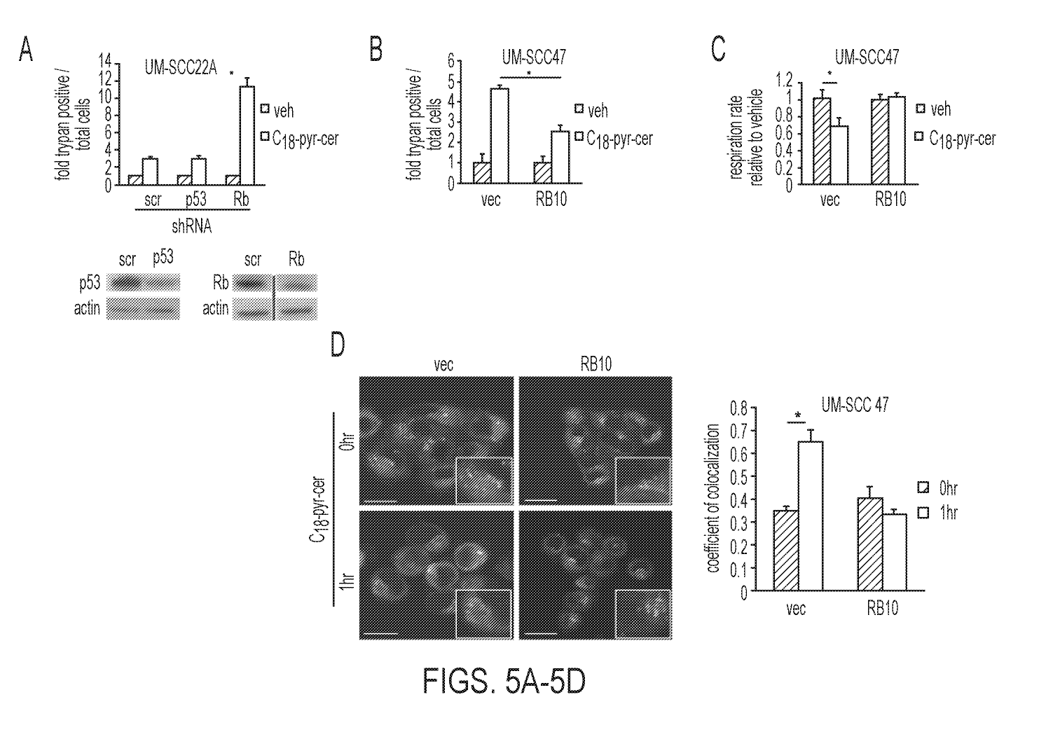

To further confirm that HPV-E7, but not HPV-E6, plays an important role in sensitizing HNSCC to C₁₈-pyr-cer, the E6 target p53 or the E7 target RB were knocked down in HPV-negative cells and their effects on cell death were measured. The data demonstrated that shRNA-mediated knockdown of RB, but not p53, increased C₁₈-pyr-cer-induced cell death (FIG. 5A) compared to Scr-shRNA-transfected and vehicle treated controls. Next, a vector was utilized to ectopically express a mutant RB (RB10), which is catalytically active but has a mutation in the E7 binding domain (L-X-C-X-E) so that it remains active in E7-expressing HPV(+) cells (Dick et al, 2000). Ectopic expression of RB10 in UM-SCC-47 cells attenuated cell death, and prevented mitophagy (measured by decreased oxygen consumption rate, and increased co-localization of LTG and MTR) in response to C₁₈-pyr-cer compared to controls (FIG. 5B-D). Thus, these data suggest that inhibition of RB signaling by HPV-E7 is key for regulator of ceramide-dependent lethal mitophagy.

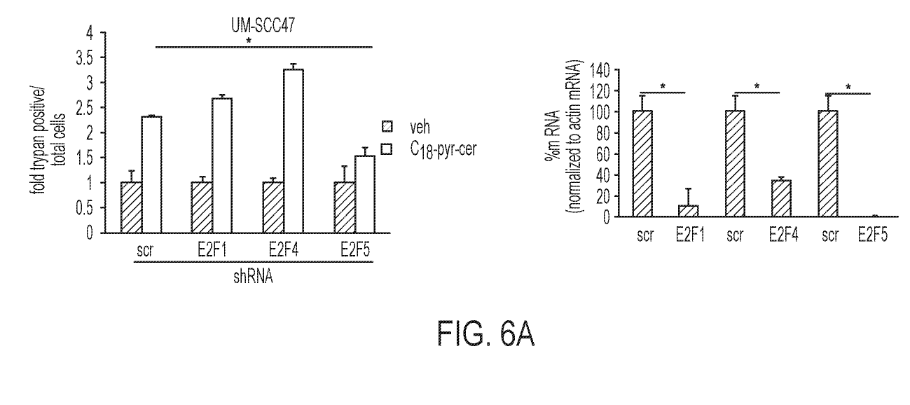

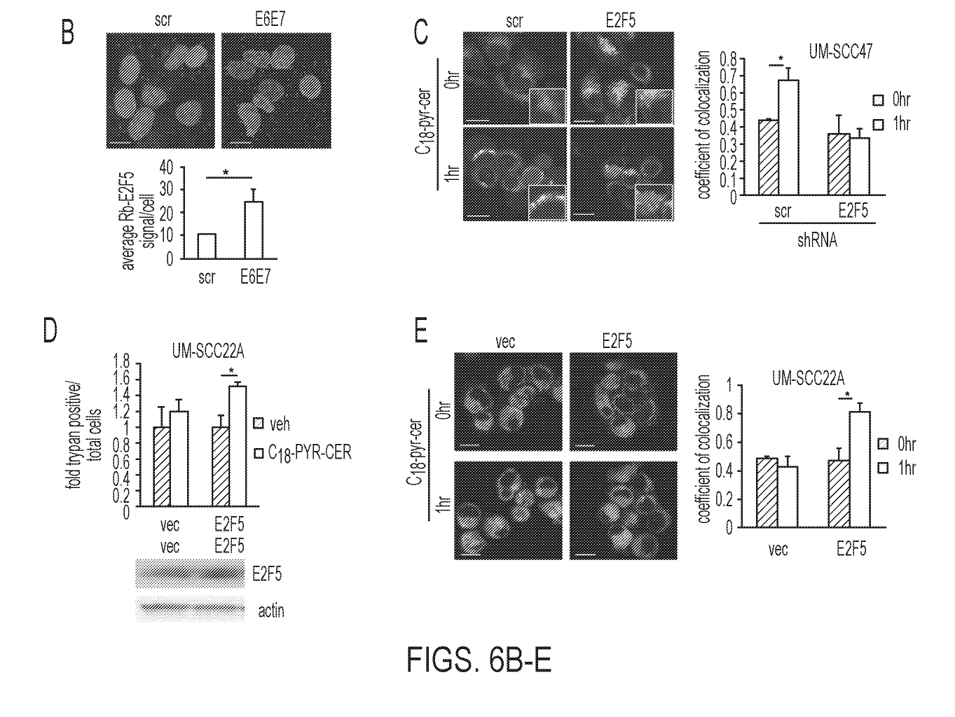

Activation of E2F5 via inhibition of RB by HPV-E7 enhances ceramide-induced mitophagy. To identify the downstream mediators of HPV-E7/RB signaling in enhancing ceramide-mediated lethal mitophagy, the roles of E2F proteins, the canonical downstream targets of RB, were investigated. Because E2F1-5 proteins have been associated with autophagy, cell death, or inhibition of cell growth previously (Morales et al, 2014; Jiang et al, 2010; Polager et al, 2008), the effects of shRNA-mediated knockdown of E2F1, E2F4, or E2F5 on ceramide-induced mitophagy were assessed. The data showed that knockdown of E2F5, but not E2F1 or E2F4, attenuated cell death in response to C₁₈-pyr-cer in HPV(+) HNSCC cells (FIG. 6A, left panel). Efficiency of shRNA-mediated knockdown of E2F1, E2F4 and E2F5 mRNAs compared to Scr-shRNA-transfected cells was confirmed using RT-PCR (FIG. 6A, right panel). The involvement of E2F5 in HPV-E7-mediated mitophagy was also consistent with a strong association between RB and E2F5, measured by proximity ligation assay (PLA) using fluorescently labeled anti-RB and anti-E2F5 antibodies, which was enhanced by knockdown of HPV-E6/E7 (FIG. 6B). Moreover, shRNA-mediated knockdown of E2F5 attenuated mitophagy (colocalization of LTG and MTR) in response to C₁₈-pyr-cer in HPV(+) UM-SCC-47 cells (FIG. 6C). Conversely, expression of exogenous E2F5 (confirmed by Western blotting, FIG. 6D, lower panel) in HPV(−) UM-SCC-22A cells enhanced cell death and increased mitophagy (colocalization of LTG and MTR) in response to C₁₈-pyr-cer compared to vector-only-transfected and vehicle-treated controls (FIG. 6D-E). Thus, these data suggest a role for E2F5 activation via the inhibition of RB by HPV-E7 in enhancing ceramide-mediated lethal mitophagy.

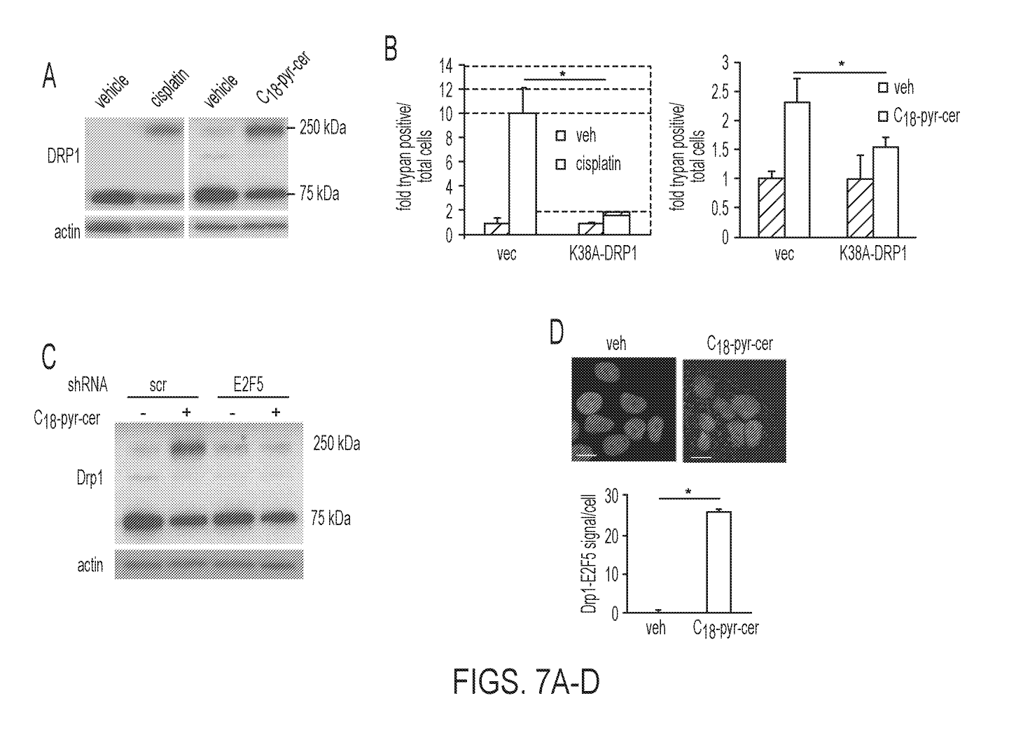

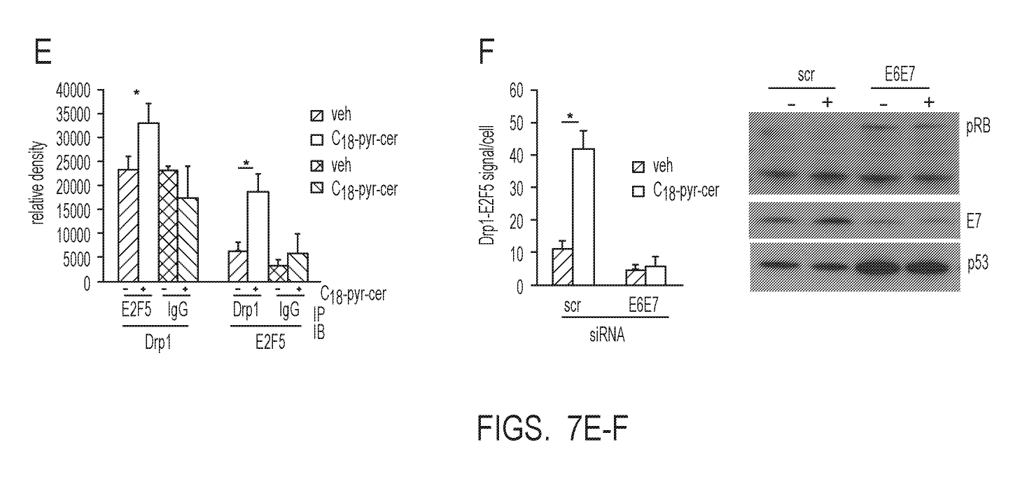

HPV-E7/ceramide-mediated mitophagy is induced by E2F5-Drp1 complex. To further define the mechanism by which E2F5 enhances HPV-E7/ceramide-mediated mitophagy, the involvement of dynamin related protein 1 (Drp1) in this process was investigated, due to the increased mitochondrial fission observed in TEM micrographs containing images of C₁₈-pyr-cer-treated UM-SCC-47 cells (FIG. 3B, bottom right image). Drp1 oligomerization and/or activation was observed in HPV(+) cells treated with cisplatin or C₁₈-pyr-cer (FIG. 7A). Expression of an inactive/dominant-negative mutant of Drp1 (K38A) inhibited cell death in response to cisplatin or C₁₈-pyr-cer compared to vector-transfected and vehicle-treated controls (FIG. 7B-C). ShRNA-mediated knockdown of E2F5 prevented C₁₈-pyr-cer-induced Drp1 oligomerization/activation compared to Scr-shRNA-transfected and vehicle-treated controls (FIG. 7C). Thus, these data suggest that Drp1 plays a key role in HPV-E7/ceramide-mediated lethal mitophagy.

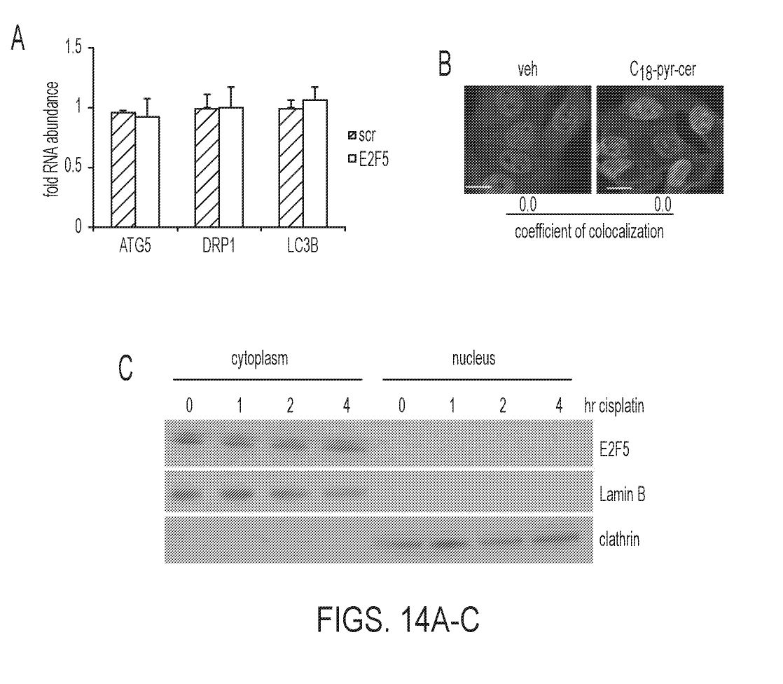

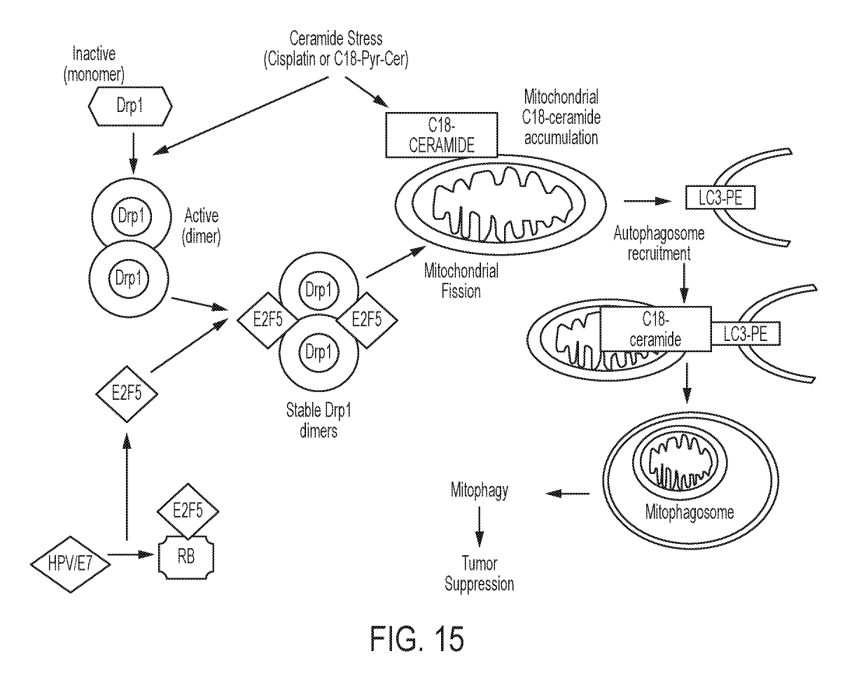

Interestingly, the data also demonstrated that alterations of E2F5 abundance by shRNA transfections had no effect on Drp1, LC3 or ATGS mRNAs compared to controls (FIG. 14A), suggesting that canonical transcription factor function of E2F5 in HPV-E7/ceramide-mediated mitophagy might be dispensable. This was supported by the cytoplasmic localization of E2F5 in the absence/presence of C₁₈-pyr-cer in HPV(+) UM-SCC-47 cells (FIGS. 14B-C). These data suggest a novel hypothesis that cytoplasmic E2F5 might associate with Drp1 for activation, leading to increased mitochondrial fission in response to ceramide stress in HPV(+) HNSCC cells. Indeed, increased Drp1-E2F5 association was detected in response to C₁₈-pyr-cer compared to controls, measured by PLA using fluorescently labeled anti-Drp1 and anti-E2F5 antibodies (FIG. 7D). Co-immunoprecipitation experiments in HPV-positive UM-SCC-47 cells treated with vehicle control or C₁₈-pyr-cer confirmed increased association between Drp1 and E2F5 (FIG. 7E). Knockdown of E6/E7 attenuated Drp1-E2F5 association (detected by PLA) compared to Scr-siRNA-transfected HPV(+) UPI-SCC-90 cells (FIG. 7F). Knockdown of HPV-E7 was confirmed by increased pRB and decreased E7 protein abundance, and knockdown of HPV-E6 was confirmed by increased p53 protein abundance compared to controls in UPI-SCC-90 cells (FIG. 7F, right panel). Thus, these data suggest a non-canonical cytoplasmic function for E2F5 to associate with Drp1 upon ceramide stress in response to C₁₈-pyr-cer or cisplatin, to induce HPV-E7-mediated lethal mitophagy.

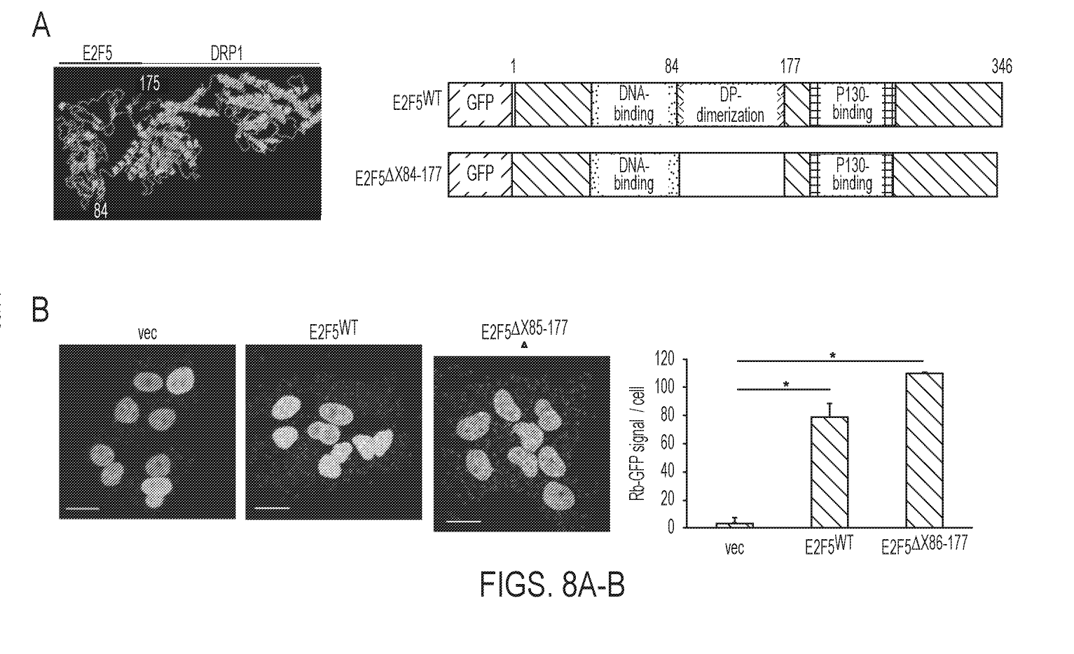

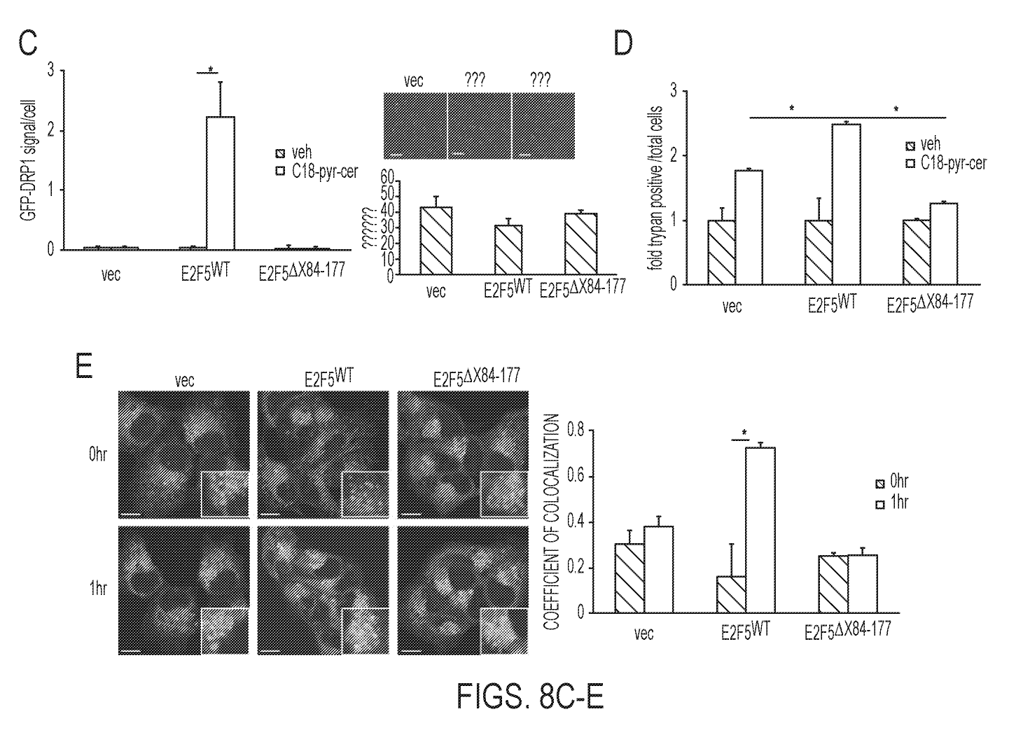

To determine the molecular details of Drp1-E2F5 complex, a specific domain of E2F5 was identified involved in Drp1 interaction by molecular docking using the ZDOCK server. These data suggested that the association between Drp1 and E2F5 might involve the “dimerization domain” of E2F5, where E2F5 is known to bind the activating dimerization partner (DP) protein, between residues 84 and 177 (FIG. 8A) (Apostolova et al, 2002). To validate these studies, a mutant E2F5 was generated, in which the dimerization domain was deleted E2F5^{Δ84-177}-GFP. To verify the integrity and correct folding of E2F5^{Δ84-177}-GFP compared to E2F5^{WT}-GFP, their association with RB by PLA was measured using labeled anti-GFP and anti-RB antibodies compared to WT-E2F5-GFP-RB association. The data demonstrated that E2F5^{Δ84-177}-GFP binds RB as effectively as E2F5^{WT} (FIG. 8B). Importantly, deletion of the dimerization domain of E2F5 almost completely inhibited the association between Drp1 and E2F5^{Δ84-177}-GFP whereas ectopically expressed E2F5^{WT}-GFP showed increased Drp1-E2F5^{WT} interaction in HPV(−) cells, measured by PLA (FIG. 8C, left panel). Comparable transfection efficiencies of GFP-tagged E2F5 proteins compared to GFP-alone (vector control) were measured by immunofluorescence in these cells (FIG. 8C, right panel). Ectopic expression of E2F5^{WT}, but not E2F5^{Δ84-177}, enhanced ceramide-induced cell death in HPV(−) cells (FIG. 8D), and increased mitophagy (as measured by colocalization of LTG and MTR) (FIG. 8E). Thus, these data suggest that the dimerization domain of E2F5 within amino acids 84-177 is involved in Drp1 association, leading to increased mitochondrial fission and ceramide-dependent mitophagy by HPV-E7 signaling.

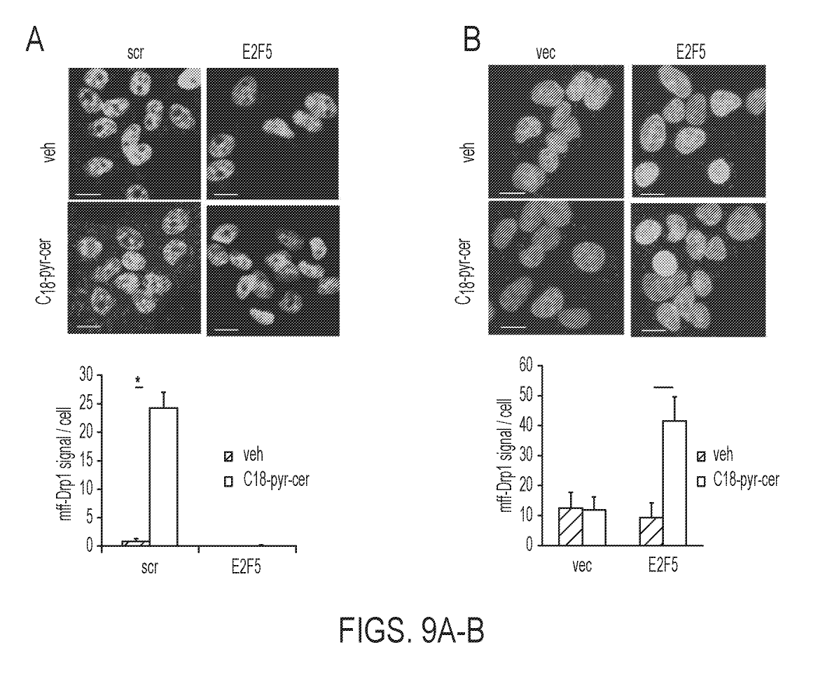

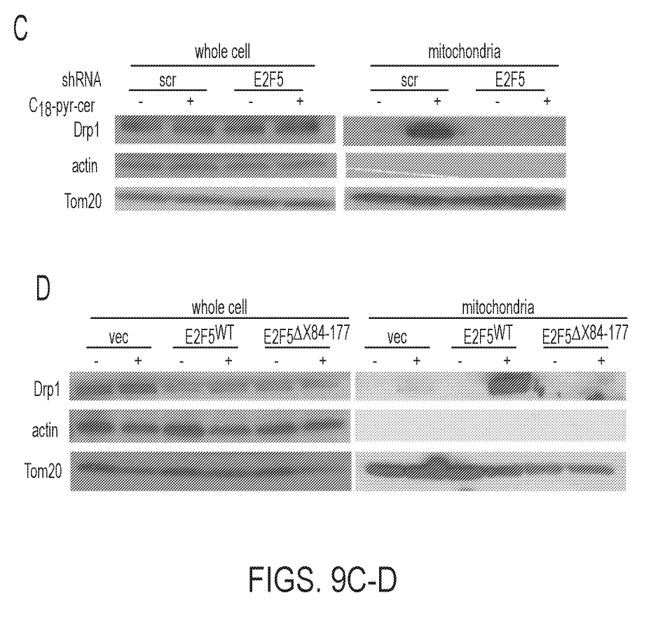

It is known that Drp1 engages with its mitochondrial receptor MFF to induce mitochondrial fission and mitophagy (Koirala et al, 2013). Thus, to determine the mechanism by which E2F5-Drp1 association enhances HPV-E7/ceramide-induced lethal mitophagy, the mitochondrial localization of Drp1 and its association with mitochondrial receptor MFF were examined. The data showed that C₁₈-pyr-cer induced Drp1-MFF association, which was attenuated by stable knockdown of E2F5 in UM-SCC47 cells compared to controls (FIG. 9A). Conversely, ectopic expression of E2F5^{WT} in HPV-negative UM-SCC-1A cells enhanced Drp1-MFF association in response to C₁₈-pyr-cer compared to vector-only-transfected and vehicle-treated controls (FIG. 9B). Moreover, C₁₈-pyr-cer induced recruitment of Drp1 to mitochondria, measured by Western blotting using mitochondria enriched subcellular fractions, which was attenuated in response to stable E2F5 knockdown in HPV(+) cells compared to Scr-shRNA-expressing cells (FIG. 9C). Reconstitution of E2F5^{WT} , but not the E2F5^{Δ84-177} mutant in HPV(+) cells, which express stable shRNAs against endogenous E2F5, rescued Drp1 recruitment to mitochondria in response to ceramide-stress compared to vector-transfected controls (FIG. 9D). Thus, these data suggest that E2F5 enhances HPV-E7/ceramide-mediated mitophagy by inducing activation and recruitment of Drp1 to mitochondria for MFF-mediated mitochondrial fission.

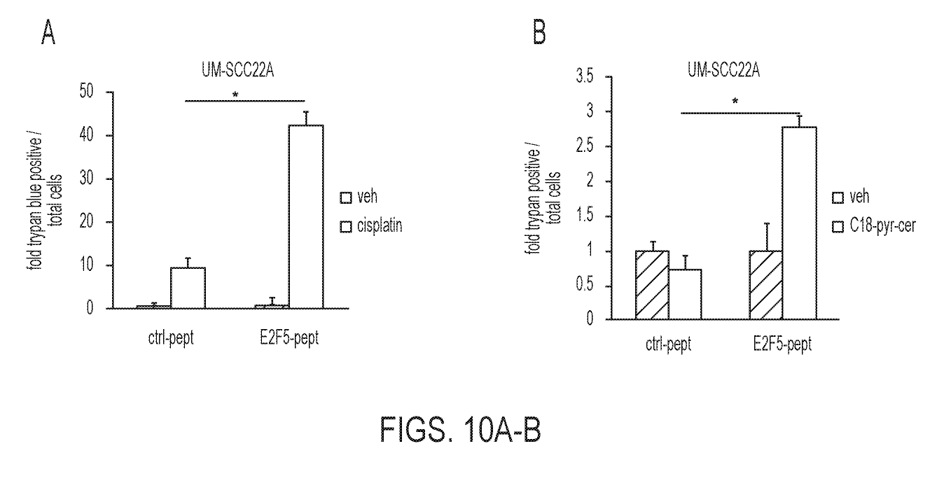

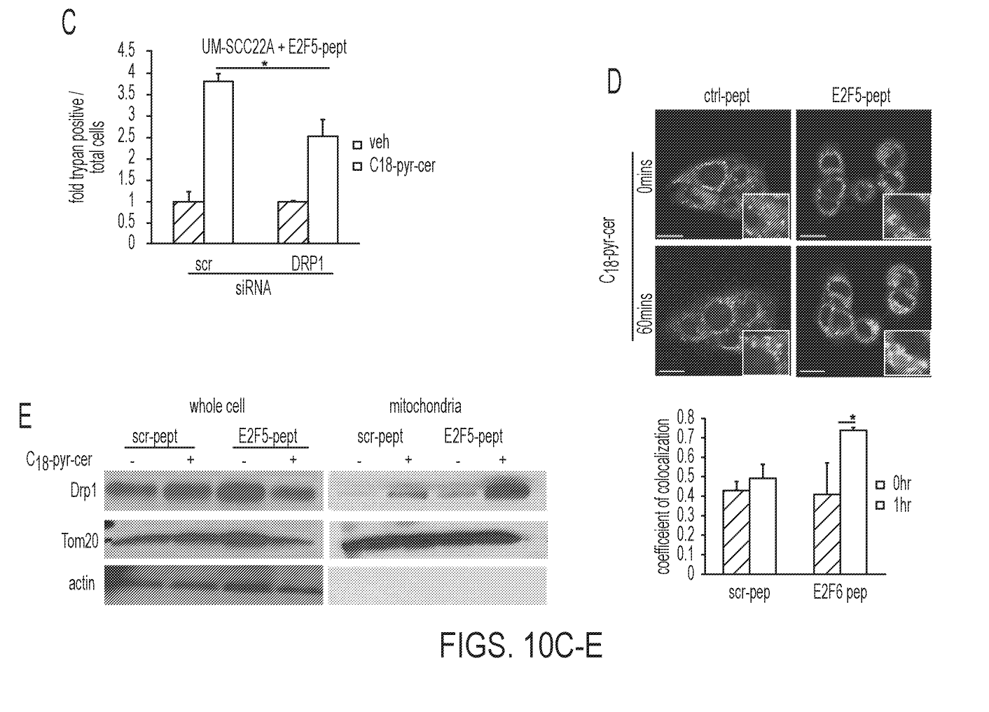

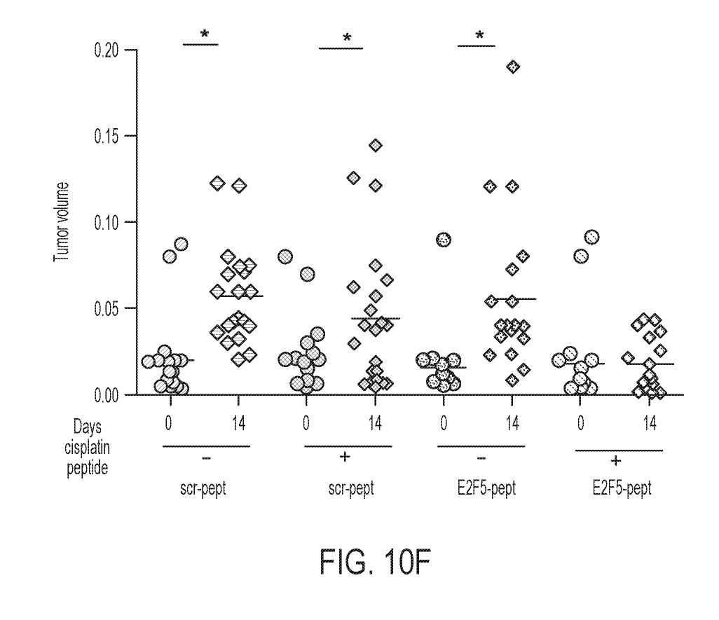

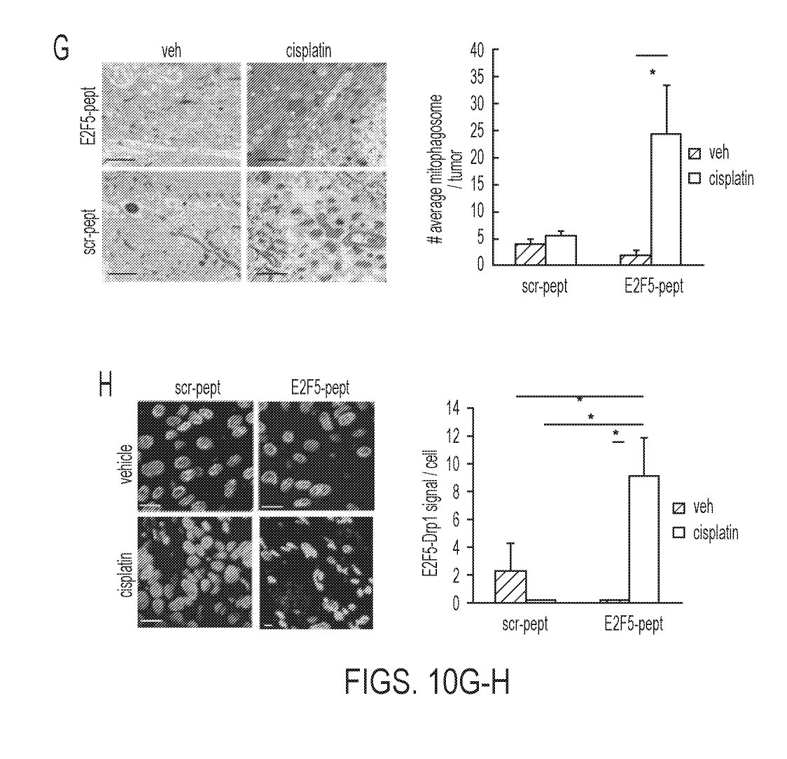

Reconstitution of E2F5-Drp1 association by E2F5-peptide mimetic enhances mitophagy in HPV(−) cells. To determine whether the putative Drp1-binding domain of E2F5 alone was sufficient to reconstitute E2F5 activity for mitophagy induction in HPV-negative cells, a peptide based on the minimal stretch of amino acids was generated, corresponding to Drp1 binding E2F5 residues (146-175, biotin-RRRRRRRR-ELDQQKLWLQQSIKNVMDDSINNRFSYVTHED (SEQ ID NO. 2)) and scrambled control peptide, which contains the same amino acids as E2F5-pept in randomized/scrambled order (biotin-RRRRRRRR-LILFVIKLHQDVNDMRNSNQDQTQSE-DRESKWY (SEQ ID NO. 3)), as predicted by molecular docking studies. Eight arginine residues (R8) were included on the N-terminus to enhance cell penetration of the E2F5 peptide (Rancher and Ryu, 2015). Importantly, treatment of UM-SCC-22A cells with the E2F5-pept largely increased cisplatin or C₁₈-pyr-cer-induced cell death compared to scr-pept (FIG. 10A-B), which was attenuated by shRNA-mediated Drp1 knockdown (FIG. 10C). E2F5-pept also enhanced C₁₈-pyr-cer-induced mitophagy, measured by increased colocalization of LTG and MTR (FIG. 10D). Moreover, E2F5-pept but not scr-pept enhanced ceramide-induced Drp1 recruitment to mitochondria in HPV(−) cells (FIG. 10E). To assess whether increased E2F5-mediated Drp1 activation increases cisplatin-mediated tumor suppression, HPV(−) UM-SCC-22A-derived xenograft tumors in SCID mice were generated, and the effects of E2F5-pept or scr-pept on tumor growth were measured in the absence/presence of cisplatin (3.5 mg/kg every 3 days for 2 weeks). Interestingly, treatment with E2F5-pept almost completely inhibited tumor growth while control peptide treatment had no effect (FIG. 10F). These data were consistent with increased mitophagy (measured by TEM) and co-localization of Drp1 and E2F5 (measured by PLA) in tumors treated with E2F5-pept in response to cisplatin compared to control tumors treated with Scr-pept with/without cisplatin (FIG. 10G-H). Thus, these data suggest that induction of E2F5-Drp1 complex using E2F5-pept is associated with decreased tumor growth in response to cisplatin compared to controls (tumors isolated from Scr-pept/vehicle-, Scr-pept/cisplatin- and/or E2F5-pept/vehicle-treated animals) in HPV(−) HNSCC-xenograft-derived tumors in SCID mice. Thus, these results suggest that the Drp1-binding domain of E2F5 is sufficient to induce mitochondrial localization of Drp1, resulting in enhanced mitochondrial fission and ceramide-dependent lethal mitophagy without the expression of HPV-E7 in HNSCC in situ and in vivo.

Example 2—Materials and Methods

Reagents. C₁₈-pyridium-ceramide was synthesized at the synthetic Lipidomics Core at the Medical University of South Carolina (MUSC). Cisplatin was purchased from Sigma. Treatments were performed using 40 μM cisplatin in DMSO, 20 μM C₁₈-pyr-cer in EtOH for 1-4 h for mitophagy detection, or corresponding amount of vehicle control. Peptides were synthesized by LifeTein, Inc. Peptides contained C-terminal amidation. E2F5-pept: Biotin-RRRRRRRR-ELDQQKLWLQQSIKNVMDDSINNRFSYVTHED (SEQ ID NO. 2). Scr-pep: Biotin-RRRRRRRR-LILFVIKLHQDVNDMRNSNQDQTQSEDRESKWY (SEQ ID NO. 3).

Antibodies used were as follows: TOM20—(F-10) Santa Cruz (sc)-17764; Actin-Sigma Rb A2066; HPV16E6-sc-1584 (N-17); HPV16E7—sc-65711 (NM2); pRB—BDBiosciences 554136 (G3-254); p53—BDBiosciences 554294 clone DO-7; CerS1 (Lass1)—(C-14) sc-65096; Ceramide—(MID 15B4) ALX-804-196-T050; E2F5—sc-999, Drp1—BD Biosciences.

Cell lines and culture conditions. HPV(+) cell lines were provided by Drs. Tom Carey, University of Michigan (UM-SCC-47) and Susan Gollin, University of Pittsburgh Cancer Institute (UPI-SCC-90). UM-SCC-22A, UM-SCC-1A, and UM-SCC-47 were grown in DMEM (Corning) with 10% FBS (Atlanta Biologicals) and 1% Penicillin and streptomycin (Cellgro) at 37° C. with 5% CO₂. UPI-SCC-90 were grown in DMEM with 10% FBS (Atlanta Biologicals), 2mM L-glutamine, 1X non-essential amino acids solution, and 500 ug/ml gentamicin (Gibco).

Stable shRNA-mediated knockdown of E2F5. pLK0.1 plasmids expressing shRNA to E2F5 or scrambled control (MUSC shRNA Shared Technology Resource) were co-transfected with pCMV-psPAX2 and pMD2 plasmids in Plat A cells using the viral transduction protocol as described by the RNA interference (RNAi) Consortium. Viral supernatants were added to UM-SCC47 cells, and selection was performed using puromycin (1 μg/ml) for 14 days.

Cell transfections. Plasmids containing target gene, shRNA, or empty vector were transfected into cells using effectene transfection reagent (QIAGEN) following the manufacturer's instructions, followed by PBS rinse six hours after transfection. CerS6, Drp1, p53, Rb, and E2F1, 4, and 5 shRNAs were from the MUSC shRNA Shared Technology Resource. siRNA transfections were performed using DharmaFECT™ (ThermoScientific, Dharmacon). SiRNAs used: E6/E7 (ThermoScientific, custom sequence AGGAGGAUGAAAUAGAUGGUU (SEQ ID NO. 4)); CerS1 , ATG5, LC3B (Thermo Scientific, Dharmacon); or non-targeting scrambled siRNA (Qiagen).

Trypan blue exclusion assay. Cells were seeded in 6-well plates, and allowed to adhere for 20 h. After treatments (for 24 h), medium containing dead cells was pelleted with trypsinized cells, then re-suspended in 1× PBS then counted in a hemocytometer after addition of trypan blue dye (Sigma-Aldrich) at a 1:10 dilution.

IC₅₀ determination by MTT assay. Cells were plated in 96-well plates and allowed to adhere for 20 h, then treated with the indicated concentrations of drug. After 48 h treatment, the MTT assay (ATCC) was performed as described previously (Sentelle et al, 2012).

Immunoblotting. Cells were lysed in RIPA buffer plus protease inhibitor cocktail on ice for 15 minutes then centrifuged. 30ug of protein from the supernatant were run on Criterion™ TGX™ Precast Gels (Bio-Rad).

Quantitative RT-PCR. RNA was extracted from cell pellets using RNeasy kit (Qiagen) per the manufacturer's instructions. cDNA was generated using equal amounts of RNA from each sample and iScript cDNA synthesis kit (Bio-Rad) per manufacturer's instructions. Reactions were carried out using SsoFast probes mix (Bio-Rad) and TaqMan primer probes (ThermoFisher Scientific) in a StepOne Plus qPCR cycler as described by the manufacturer.

Site-directed mutagenesis. E2F5^{Δ84-177} mutant was generated using Q5 site-directed mutagenesis kit (New England Biolabs) per manufacturer's instructions. Primers were designed using NEBaseChanger (New England Biolabs). Forward: GAG GTG GAG GTC TAG ATC ACC AAT GTC TTA GAG GG (SEQ ID NO. 5), Reverse: CAG TGT GGT GGA ATT CTA TGT ATC ACC ATG AAA GC (SEQ ID NO. 6).

Molecular modeling of protein-protein interactions. Phyre2 was used to predict secondary structure of E2F5 based on the sequence in GenBank (NP_001942.2). The generated PDB file was input along with the PDB for Drp1(4BEJ) from RSCB (www.rscb.org) into ZDOCK Server (http://zdock.umassmed.edu/). The top model was then used to predict the sites of association between E2F5 and Drp1.

Measurement of cellular respiration using Seahorse XF analyzer. Cells were plated in a Seahorse Biosciences 96-well plate and allowed to adhere for 20 hours. They were then treated with 20 uM C₁₈-pyr-cer or equivalent amount of vehicle (EtOH) for two hours, then oxygen consumption rate was measured using a Seahorse XF96 (Seahorse Biosciences) as described by the manufacturer (Sentelle et al, 2012).

Proximity ligation assay. Proximity ligation assays were performed using Duolink in situ red kit (Sigma) per manufacturer's instructions, then analyzed as described (Panneer-Selvam et al, 2015).

Immunofluorescence. Cells were plated on glass coverslips in 6-well plates and allowed to adhere for 20 hours. Treatment was performed with 40 uM cisplatin or equivalent amount of DMSO for 8 hr, or 5 μM peptide for 2 hours. Fixation was in 4% paraformaldehyde, followed by permeabilization with 0.1% Triton-X100, and blocking in 1% BSA in PBS. Samples were incubated at 4° C. overnight with primary antibodies in blocking solution. TOM20 (SCBT) 1:200, Lass1/CerS1 (SCBT) 1:50, ceramide (Enzo Life Sciences) 1:100, and biotin (SCBT) 1:200. Immunofluorescent-conjugated secondary antibodies (AlexaFluor 488 or 594, Jackson Immuno) were added at 1:500 for one hour. Coverslips were then mounted onto glass slides with ProLong® Gold Antifade Mountant (Molecular Probes).

Laser scanning confocal microscopy. For live cell imaging, cultured cells were incubated with 500 nM of mitotracker far red and 500 nM lysotracker green in DMSO for 30 min at 37° C. Cells were treated with 20 uM C₁₈-pyr-cer or 40 uM cisplatin and kept in an incubator with 5% CO₂ at 37° C. during imaging. An Olympus FV10i confocal microscope was used for imaging. 543- and 488-nm channels were used for visualizing red or green fluorescence, respectively, with pinholes set to 1.0 Airy units. At least three random fields were imaged for each sample (Sentelle et al, 2012).

Ultra-structure analysis using transmission electron microscopy. Cells were washed with 1× PBS then fixed in 2% glutaraldehyde (w/v) in 0.1 M cacodylate. After post-fixation in 2% (v/v) osmium tetroxide, specimens were embedded in Epon 812, and sections were cut orthogonally to the cell monolayer with a diamond knife. Thin sections were visualized in a JEOL 1010 transmission electron microscope (Saddoughi et al, 2013).

Cell fractionation. Cells were treated with 40 uM cisplatin or vehicle for eight hours, 20 uM C₁₈-pyr-cer or vehicle and/or 5 uM scr-pept or E2F5-pept for 1.5 hours. Mitochondria isolation kit (ThermoFisher Scientific) was used as described by the manufacturer.

Co-immunoprecipitation. Cells were lysed in 500 ul Pierce™ IP lysis/wash buffer (ThermoFisher) with protease inhibitor cocktail (Sigma) on ice for 15 minutes. 350 μg of protein were used with SureBeads™ Protein A Magnetic Beads (BioRad) per manufacturer's instructions, using 10 μg antibody or corresponding normal IgG control (SCBT).

Image Quantification. Images were quantified by ImageJ. Co-localization in micrographs was measured using FIJI. Duolink ImageTool software was used for quantification of PLA signals (Panneer-Selvam et al, 2015).

In vivo studies. Severe combined immunodeficient (SCID) mice were purchased from Jackson Laboratories. Age- and sex-matched mice were used. All animal protocols were approved by the Institutional Animal Care and Use Committee at the Medical University of South Carolina. UM-SCC22A or UM-SCC47 cells (75,000) were implanted into the flanks of SCID mice (n=5-8 mice). When the tumors were palpable, the mice were treated every three days with 3.5 mg/kg cisplatin, 20 mg/kg C₁₈-pyr-cer, or corresponding amount of vehicle control and/or 3.76 μg E2F5-peptide or scrambled control peptide. Tumor volume was measured using calipers. At the end of the 14-day treatment, the mice were euthanized and tumor tissues were collected (Sentelle et al, 2007; Saddoughi et al, 2013).

Statistical analysis. Data were reported as mean±standard error. Mean values were compared using the student t test and p<0.05 was considered statistically significant (Saddoughi et al, 2013).

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

Ambrus A M, Islam A B, Holmes K B, Moon N S, Lopez-Bigas N, Benevolenskaya E V, Frolov M V (2013) Loss of dE2F compromises mitochondrial function. Dev Cell 27: 438-451

Ang K K, Harris J, Wheeler R, Weber R, Rosenthal D I, Nguyen-Tan P F, Westra W H, Chung C H, Jordan R C, Lu C et al (2010) Human papillomavirus and survival of patients with oropharyngeal cancer. N Engl J Med 363: 24-35

Apostolova M D, Ivanova I A, Dagnino C, D'Souza S J, Dagnino L (2002) Active nuclear import and export pathways regulate E2F-5 subcellular localization. J Biol Chem 277: 34471-34479

Bol V, Gregoire V (2014) Biological basis for increased sensitivity to radiation therapy in HPV-positive head and neck cancers. Biomed Res Int 2014: 1-6

Chang W Y, Bryce D M, D'Souza SJA, Dagnino L (2004) The DP-1 transcription factor is required for keratinocyte growth and epidermal stratification. J Biol Chem 279: 51343-51353

Chen H Z, Tsai S Y, Leone G (2009) Emerging roles of E2Fs in cancer: an exit from cell cycle control. Nat Rev Cancer 9: 785-797

Cosway B, Lovat P (2016) The role of autophagy in squamous cell carcinoma of the head and neck. Oral Oncol 54: 1-6

Dany M, Ogretmen B (2015) Ceramide induced mitophagy and tumor suppression. Biochim Biophys Acta-Mol Cell Res 1853: 2834-2845

Dick F A, Sailhamer E, Dyson N J (2000) Mutagenesis of the pRB pocket reveals that cell cycle arrest functions are separable from binding to viral oncoproteins. Mol Cell Biol 20: 3715-3727

D'Souza G, Kreimer A R, Viscidi R, Pawlita M, Fakhry C, Koch W M, Westra W H, Gillison M L (2007) Case-control study of human papillomavirus and oropharyngeal cancer. N Engl J Med 356: 1944-1956

Dyson N, Howley P M, Munger K, Harlow E (1989) The human papilloma virus-16 E7 oncoprotein is able to bind to the retinoblastoma gene product. Science 243: 934-937

Fakhry C, Westra W H, Li S, Cmelak A, Ridge J A, Pinto H, Forastiere A, Gillison M L (2008) Improved survival of patients with human papillomavirus-positive head and neck squamous cell carcinoma in a prospective clinical trial. J Natl Cancer Inst 100: 261-269

Garcia-Zepeda S P, Garcia-Villa E, Diaz-Chavez J, Hernandez-Pando R, Gariglio P (2013) Resveratrol induces cell death in cervical cancer cells through apoptosis and autophagy. Eur J Cancer Prey 22: 577-584

Gemin A, Sweet S, Preston T J, Singh G (2005) Regulation of the cell cycle in response to inhibition of mitochondrial generated energy. Biochem Biophys Res Commun 332: 1122-1132

Halbert C L, Demers G W, Galloway D A (1992) The E6 and E7 genes of human papillomavirus type 6 have weak immortalizing activity in human epithelial cells. J Virol 66: 2125-2134

Hanada K, Kumagai K, Yasuda S, Miura Y, Kawano M, Fukasawa M, Nishijima M (2003) Molecular machinery for non-vesicular trafficking of ceramide. Nature 426: 803-809

Helt A M, Funk J O, Galloway D A (2002), Inactivation of both the retinoblastoma tumor suppressor and p21 by the human papillomavirus type 16 E7 oncoprotein is necessary to inhibit cell cycle arrest in human epithelial cells. J Virol 76: 10559-10568

Hijmans E M, Voorhoeve P M, Beijersbergen R L, van 't Veer U, Bernards R (1995) E2F-5, a new E2F family member that interacts with p130 in vivo. Mol Cell Biol 15: 3082-3089

Itoh A, Levinson S F, Morita T, Kourembanas S, Brody J S, Mitsialis S A (1995) Structural characterization and specificity of expression of E2F-5: a new member of the E2F family of transcription factors. Cell Mol Biol Res 41: 147-154

Jiang H, Martin V, Gomez-Manzano C, Johnson D G, Alonso M, White E, Xu J, McDonnell T J, Shinojima N, Fueyo J (2010) The RB-E2F1 pathway regulates autophagy. Cancer Res 70,7882-7893

Killock D (2015) Therapeutic HPV vaccine holds promise. Nat Rev Clin Oncol 12: 686

Koirala S, Guo Q, Kalia R, Bui H T, Eckert D M, Frost A, Shaw J M (2013) Interchangeable adaptors regulate mitochondrial dynamin assembly for membrane scission. Proc Natl Acad Sci USA 110: E1342-E1351

Koybasi S, Senkal C E, Sundararaj K, Spassieva S, Bielawski J, Osta W, Day T A, Jiang J C, Jazwinski S M, Hannun Y A (2004) Defects in cell growth regulation by C18:0-ceramide and longevity assurance gene 1 in human head and neck squamous cell carcinomas. J Biol Chem 279:44311-44319

Lee B K, Bhinge A A, Iyer V R (2011) Wide-ranging functions of E2F4 in transcriptional activation and repression revealed by genome-wide analysis. Nucleic Acids Res 39: 3558-3573

Leemans C R, Braakhuis B J, Brakenhoff R H (2011) The molecular biology of head and neck cancer. Nat Rev Cancer 11: 9-22

Lehtinen M, Dillner J (2013) Clinical trials of human papillomavirus vaccines and beyond. Nat Rev Clin Oncol 10: 400-410

Mirghani H, Amen F, Tao Y, Deutsch E, Levy A (2015) Increased radiosensitivity of HPV-positive head and neck cancers: Molecular basis and therapeutic perspectives. Cancer Treat Rev 41: 844-852

Moody C A, Fradet-Turcotte A, Archambault J, Laimins L A (2007) Human papillomaviruses activate caspases upon epithelial differentiation to induce viral genome amplification. Proc Natl Acad Sci U S A 104: 19541-19546

Morales L D, Casillas Pavan E A, Shin J W, Garcia A, Capetillo M, Kim D J, Lieman J H, Bhattacharya S (2014) Protein tyrosine phosphatases PTP-1B, SHP-2, and PTEN facilitate Rb/E2F-associated apoptotic signaling. PLoS One 9: e97104

Panneer Selvam S, De Palma R M, Oaks J J, Oleinik N, Peterson Y K, Stahelin R V, Skordalakes E, Ponnusamy S, Garrett-Mayer E, Smith C D et al (2015) Binding of the sphingolipid SIP to hTERT stabilizes telomerase at the nuclear periphery by allosterically mimicking protein phosphorylation. Sci Signal 8: ra58

Pewzner-Jung Y, Ben-Dor S, Futerman A H (2006) When do Lasses (longevity assurance genes) become CerS (ceramide synthases)?: Insights into the regulation of ceramide synthesis. J Biol Chem 281: 25001-25005

Polager S, Ofir M, Ginsberg D (2008) E2F1 regulates autophagy and the transcription of autophagy genes. Oncogene 27: 4860-4864

Sadasivam S, DeCaprio J A (2013) The DREAM complex: master coordinator of cell cycle-dependent gene expression. Nat Rev Cancer 13: 585-595

Saddoughi S A, Gencer S, Peterson Y K, Ward K E, Mukhopadhyay A, Oaks J, Bielawski J, Szulc Z M, Thomas R J, Panneer Selvam S et al (2013) Sphingosine analogue drug FTY720 targets I2PP2A/SET and mediates lung tumour suppression via activation of PP2A-RIPK1-dependent necroptosis. EMBO Mol Med 5: 105-121

Scheffner M, Werness B A, Huibregtse J M, Levine A J, Howley P M (1990) The E6 oncoprotein encoded by human papillomavirus types 16 and 18 promotes the degradation of p53. Cell 63: 1129-1136

Senkal C E, Ponnusamy S, Rossi M J, Sundararaj K, Szulc Z, Bielawski J, Bielawska A, Meyer M, Cobanoglu B, Koybasi S (2006) Potent antitumor activity of a novel cationic pyridinium-ceramide alone or in combination with gemcitabine against human head and neck squamous cell carcinomas in vitro and in vivo. J Pharmacol Exp Ther 317: 1188-1199

Sentelle R D, Senkal C E, Jiang W, Ponnusamy S, Gencer S, Panneer Selvam S, Ramshesh V K, Peterson Y K, Lemasters J J, Szulc Z M (2012) Ceramide targets autophagosomes to mitochondria and induces lethal mitophagy. Nat Chem Biol 8: 831-838

Smirnova E, Griparic L, Shurland D L, van der Bliek A M (2001) Dynamin-related protein Drp1 is required for mitochondrial division in mammalian cells. Mol Biol Cell 12: 2245-2256

Spassieva S D, Ji X, Liu Y, Gable K, Bielawski J, Dunn T M, Bieberich E, Zhao L (2016) Ectopic expression of ceramide synthase 2 in neurons suppresses neurodegeneration induced by ceramide synthase 1 deficiency. Proc Natl Acad Sci USA 113: 5928-5933

Strack S, Cribbs J T (2012) Allosteric modulation of Drp1 mechanoenzyme assembly and mitochondrial fission by the variable domain. J Biol Chem 287: 10990-11001 Raucher D, Ryu J S (2015) Cell-penetrating peptides: strategies for anticancer treatment. Trends Mol Med 21: 560-570

Rieckmann T, Tribius S, Grob T J, Meyer F, Busch C J, Petersen C, Dikomey E, Kriegs M (2013) HNSCC cell lines positive for HPV and p16 possess higher cellular radiosensitivity due to an impaired DSB repair capacity. Radiother Oncol 107: 242-246

Tao Y, Kassatly R F, Cress W D (1997) Subunit composition determines E2F DNA-binding site specificity. Mol Cell Biol 17: 6994-7007

van Moorsel C J, Pinedo H M, Veerman G, Vermorken J B, Postmus P E, Peters G J (1999). Scheduling of gemcitabine and cisplatin in Lewis lung tumour bearing mice. Eur J Cancer 35: 808-814

Venkataraman K, Riebeling C, Bodennec J, Riezman H, Allegood J C, Sullards M C, Merrill Jr A H, Futerman A H (2002) Upstream of growth and differentiation factor 1 (uog1), a mammalian homolog of the yeast longevity assurance gene 1 (LAG1), regulates N-stearoyl-sphinganine (C₁₈-(dihydro)ceramide) synthesis in a fumonisin Bl-independent manner in mammalian cells. J Biol Chem 277: 35642-35649

Weinberg S E, Chandel N S (2015) Targeting mitochondria metabolism for cancer therapy. Nat Chem Biol 11: 9-15

White E A, Sowa M E, Tan M J, Jeudy S, Hayes S D, Santha S, Munger K, Harper J W, Howley P M (2012) Systematic identification of interactions between host cell proteins and E7 oncoproteins from diverse human papillomaviruses. Proc Natl Acad Sci U S A 109: E260-E267

Wu W, Lin C, Wu K, Jiang L, Wang X, Li W, Zhuang H, Zhang X, Chen H, Li S et al (2016) FUNDC1 regulates mitochondrial dynamics at the ER-mitochondrial contact site under hypoxic conditions. EMBO J: e201593102

Zhang C S, Lin SC (2016) AMPK promotes autophagy by facilitating mitochondrial fission. Cell Metab 23: 399-401

Zhao L, Spassieva S D, Jucius T J, Shultz L D, Shick H E, Macklin W B, Hannun Y A, Obeid L M, Ackerman S L (2011) A deficiency of ceramide biosynthesis causes cerebellar purkinje cell neurodegeneration and lipofuscin accumulation. PLoS Genet 7: e1002063

Ziemann F, Arenz A, Preising S, Wittekindt C, Klussmann J P, Engenhart-Cabillic R, Wittig A (2015) Increased sensitivity of HPV-positive head and neck cancer cell lines to x-irradiation and cisplatin due to decreased expression of E6 and E7 oncoproteins and enhanced apoptosis. Am J Cancer Res 5: 1017-1031

What is claimed is:

1. An isolated polypeptide comprising an amino acid sequence having at least 90% sequence identity to SEQ ID NO: 2 or SEQ ID NO: 2, wherein the polypeptide comprises no more than 100 contiguous amino acids of SEQ ID NO: 13.

2. The polypeptide of claim 1, wherein the polypeptide comprises no more than 50 contiguous amino acids of SEQ ID NO: 13.

3. The polypeptide of claim 1, wherein the polypeptide is less than 100 amino acids in length.

4. The polypeptide of claim 1, wherein the polypeptide comprises a sequence at least 90% identical to SEQ ID NO: 1.

5. The polypeptide of claim 4, wherein the polypeptide comprises an amino acid sequence having at least 95% sequence identity to SEQ ID NO:l.

6. The polypeptide of claim 1, wherein the polypeptide comprises a sequence at least 90% identical to SEQ ID NO: 2.

7. The polypeptide of claim 1, wherein the polypeptide comprises a sequence at least 95% identical to SEQ ID NO: 2.

8. The polypeptide of claim 7, wherein the polypeptide comprises an amino acid sequence of SEQ ID NO: 2.

9. The polypeptide of claim 1, further comprising a cell penetration sequence.

10. The polypeptide of claim 9, wherein the cell penetration sequence is a polyarginine sequence.

11. A pharmaceutical composition comprising the isolated polypeptide of any one of claims 1-10 and a pharmaceutical carrier.

12. The composition of claim 11, wherein the pharmaceutical composition is formulated for parenteral administration, intravenous injection, intramuscular injection, inhalation, or subcutaneous injection.

13. An isolated nucleic acid encoding the polypeptide of any one of claims 1-7.

14. The nucleic acid of claim 13, wherein the nucleic acid is comprised in a vector.

15. The nucleic acid of claim 14, wherein the vector comprises a mammalian expression vector.

16. A host cell comprising the nucleic acid of claim 13.

17. A method for treating cancer in a subject comprising administering an effective amount of the polypeptide of any one of claims 1-10 or a nucleic acid of claim 15 to the subject.

18. The method of claim 17, wherein the cancer is head and neck cancer.

19. The method of claim 17, wherein the subject is diagnosed as Human papillomavirus (HPV) negative.

20. The method of claim 17, further comprising administering at least a second anti-cancer therapy.

21. The method of claim 20, wherein the second anti-cancer therapy is selected from the group consisting of a chemotherapy, a radiotherapy, an immunotherapy, or a surgery.

22. The method of claim 21, wherein the chemotherapy is cisplatin.

23. The method of claim 20, wherein the at least a second anti-cancer therapy is a ceramide analogue drug.

24. The method of claim 23, wherein the ceramide analogue drug is C₁₈-pyridinium-ceramide (C₁₈-pyr-cer).
